(19) United States
(12) Patent Application Publication
(10) Pub. No.: US 2025/0290156 A1
(43) Pub. Date: Sep. 18, 2025

(54) EVALUATING OVARIAN CANCER CHEMOTHERAPY RESPONSE USING GENE EXPRESSION DATA AND MACHINE LEARNING

(71) Applicant: George Mason University, Fairfax, VA (US)

(72) Inventors: Mohsin Saleet Jafri, Potomac, MD (US); Soukaina Amniouel, Fairfax, VA (US)

(21) Appl. No.: 19/080,152

(22) Filed: Mar. 14, 2025

Related U.S. Application Data
(60) Provisional application No. 63/564,857, filed on Mar. 13, 2024.

(51) Int. Cl. C12Q 1/6886; G01N 33/574; G16B 40/00

(52) U.S. Cl. CPC C12Q 1/6886; G01N 33/57449; G16B 40/00; C12Q 2600/106; C12Q 2600/158

(57) ABSTRACT

The present disclosure generally relates to gene expression profiling of tissue samples obtained from ovarian cancer patients who are candidates for chemotherapy treatment. More specifically, the disclosure provides methods based on characterization of gene expression which allow a physician to predict whether a patient is likely to respond well to treatment with a chemotherapeutic reagent.

This application claims benefit to provisional application Ser. No. 63/564,857 filed on Mar. 14, 2024, the contents of which are incorporated by reference in its entirety herein.

### TECHNICAL FIELD

The present disclosure generally relates to gene expression profiling of tissue samples obtained from ovarian cancer patients who are candidates for chemotherapy treatment. More specifically, the disclosure provides methods based on characterization of gene expression which allow a physician to predict whether a ovarian cancer patient is likely to respond well to treatment with platinum-paclitaxel or platinum-only chemotherapy.

### BACKGROUND

Ovarian cancer (OC) is considered to be the most lethal gynecological cancer in the United States. At present, there is no effective screening for OC, resulting in a substantial number of cases being diagnosed at advanced stages of cancer characterized by tumor metastasis. The standard treatment consists of optimal cytoreductive surgery with subsequent treatment with a combination of platinum and taxane-based chemotherapy. In general, patients who positively respond to initial chemotherapy, classified as responders, show a favorable prognosis with a median survival rate exceeding four years. However, it has been known that one-third of HGSOC patients face disease progression or recurrence after initial treatment. These non-responders receive a second-line treatment not involving platinum agents. There is currently a need for predictive biomarkers that can effectively differentiate between the use of platinum-paclitaxel chemotherapy and platinum-only chemotherapy for SOC patients.

### SUMMARY

The present disclosure relates to the identification of gene expression levels in tissue samples obtained from patients with serious ovarian cancer (SOC) who responded or did not respond to treatment with platinum-paclitaxel or platinum-only treatment. As described herein, prognostic mRNAs, referred to herein as “biomarkers”, have been identified wherein the expression levels of said biomarkers correlated to the likelihood of responding to platinum-paclitaxel or platinum-only treatment chemotherapy. All biomarker gene symbols, as used herein, are those adopted by the HUGO Gene Nomenclature Committee. Amino acid and nucleic acid sequences corresponding to each of the listed genes are publicly available.

In one embodiment, the disclosure provides a method for predicting the likelihood that patients diagnosed with SOC, who are candidates for treatment with platinum-paclitaxel or platinum-only treatment, will respond to such treatment, comprising determining the expression level of one or more biomarker transcripts, or their expression product, in a SOC patient sample, wherein the biomarker transcript is the transcript of one or more genes selected from the group consisting of those differentially expressed genes of Table 6-7.

In one embodiment, the disclosure provides a method for predicting the likelihood that patients with SOC, who are candidates for treatment with platinum-paclitaxel chemotherapy, will respond to such treatment, comprising determining the expression level of one or more biomarker RNA transcripts, or their expression product, in a SOC patient sample, wherein the biomarker transcript is the transcript of one or more genes selected from the group consisting of ICAM1, TUBB2A, GLDC, PLAU, AURKA, NEAT1, MXRA5, GSN, and MUC16 (FIG. 5A-I).

In one embodiment, the disclosure provides a method for predicting the likelihood that patients diagnosed with SOC, who are candidates for treatment with platinum-only treatment, will respond to such treatment, comprising determining the expression level of one or more biomarker RNA transcripts, or their expression product, in a SOC patient sample, wherein the biomarker transcript is the transcript of one or more genes selected from the group consisting of: FCGBP, TFPI, NUAK1, LRRC17, FLRT2, IL12A, HSPA2, CDC20, FOXM1, and MAP4K2 (FIG. 6A-I).

The expression levels of one or more biomarker mRNA transcripts, or their protein products, can be determined by methods known in the art. Methods for detecting expression of the biomarker genes disclosed herein, include methods based on hybridization analysis of polynucleotides, methods based on sequencing of polynucleotides, immunohistochemistry methods, and proteomics-based methods. The methods generally detect expression products (e.g., mRNA or encoded proteins) of the biomarker genes. In preferred embodiments, PCR-based methods, such as reverse transcription PCR (RT-PCR), and array-based methods are used.

In one aspect, microarrays are provided comprising one or more biomarker genes that demonstrate altered expression following exposure to platinum-paclitaxel or platinum-only. In an embodiment, the microarray may comprise one or more probes representative of the biomarker genes disclosed herein wherein the expression levels of said genes correlate to a SOC patient's likelihood of responding to platinum-paclitaxel or platinum-only treatment chemotherapy. In another aspect, methods are provided for using said microarrays to provide a patient's prognosis for responding to platinum-paclitaxel or platinum-only chemotherapy through the generation of an expression profile indicating that the patient is a candidate for treatment with platinum-paclitaxel or platinum-only chemotherapy.

In an embodiment a method is provided for determining a patient's prognosis for responding to platinum-paclitaxel or platinum-only chemotherapy comprising the steps of: (i) providing a nucleic acid probe comprising a nucleotide sequence having at least 10, at least 15, at least 25 or at least 40 consecutive nucleotides complementary to the one or more biomarker RNA transcripts disclosed herein, wherein the expression levels of the one or more biomarker RNAs correlates to a SOC patient's likelihood of responding to platinum-paclitaxel or platinum-only chemotherapy; (ii) contacting the nucleic acid probe under stringent conditions with the mRNA of a patient's tissue sample; and (iii) detecting the amount of hybridization, wherein detection of a similarity in the amount of hybridization with the RNA of the patient's test sample, as compared to the amount of hybridization of a control test sample, i.e., a non-responder and/or a responder sample, is indicative of the patient's prognosis for responding to platinum-paclitaxel or platinum-only chemotherapy.

The present disclosure provides a method of preparing a prognostic profile for a SOC patient, comprising the steps of: (i) subjecting a patient's sample containing biomarker mRNA to gene expression analysis; (ii) determining the expression level of one or more of the biomarker mRNAs disclosed herein wherein the expression level is compared to control levels of expression determined for responder and non-responder samples; and (iii) creating a report summarizing the data obtained by said gene expression analysis.

The present disclosure provides a kit for identifying the expression levels of one or more of the biomarker mRNAs disclosed herein. Said kit comprises a probe/primer for detecting the level of one or more biomarker RNAs in a sample derived from a patient. In certain embodiments, the kit may further include instructions for using the kit, solutions for suspending or fixing cells derived from the sample, detectable tags or labels, and solutions for lysing cells. In some instances, the kit may contain solutions and reagents for detecting the protein products of the biomarker mRNAs.

### BRIEF DESCRIPTION OF THE FIGURES

FIG. 1. Gene expression profiling datasets of human serous ovarian cancer tissues from the NCBI-GEO database were analyzed to identify differentially expressed genes (DEGs) using the robust multi-array average method in R. The LASSO and varSelRF feature selection methods were used to identify gene signatures related to each chemotherapy drug (i.e., platinum-paclitaxel or platinum-only). The performance of random forest and support vector algorithms as the machine learning model was evaluated. Functional enrichment analysis used the IPA online tool. Progression-free survival and overall survival analysis utilized the Kaplan-Meier plotter online tool.

FIG. 2A-B. Before and after batch correction PCA clustering plot. (FIG. 2A) The PCA results before and after applying a batch correction method on serous ovarian cancer samples who received the platinum-paclitaxel drug. (FIG. 2B) The PCA results before and after applying a batch correction method on serous ovarian cancer samples who received the platinum-only drug.

FIG. 3A-B. Volcano plots showing the distribution of the gene expression fold changes in serous ovarian cancer patients who received either (FIG. 3A) platinum-paclitaxel or (FIG. 3B) platinum-only treatment. The x-axis of the plot represents the log₂ fold change in gene expression [log₂ fold change=log₂ ((X^{D}ᵢ/X^{C}ᵢ)) where X^{D}ᵢ and X^{C}ᵢ are the average intensities of the gene of responders and non-responders, respectively], indicating the direction and magnitude of change. The y-axis displays the negative logarithm of the adjusted p-value, emphasizing the statistical significance of each gene's expression difference. Red dots represent genes with a statistically significant increase or decrease in expression, indicated by a log2 fold change (log2FC) greater than 1 or less than −1 and adjusted p-value less than 0.05. Blue dots indicate genes with statistically significant adjusted p-value less than 0.5, but with a log2FC that do not reach the set cut-offs for up-or downregulation. Green dots show genes that, while not meeting the stringent criteria for up-or downregulation, display a noteworthy fold change or p-value, suggesting potential biological significance. Grey dots correspond to genes that do not meet the significance threshold for differential expression, with fold changes and p-values that do not reach the set cut-offs for up-or downregulations.

FIG. 4A-D. Identification of the relevant genes associated with ovarian cancer and platinum-based drug using LASSO. (FIG. 4A,C) The cross-validation error plots in a LASSO model. The plots provide insights into the model's performance across different levels of complexity represented by varying values of the regularization parameter, lambda. The x-axis represents the lambda values on a logarithmic scale helping to visualize the wide range of lambda values explored during the model fitting process. The error bars on the mean cross-validation error curve show the standard error for different lambda values, indicating the variability in model performance across complexities. Smaller error bars suggest greater confidence in the error estimates at those lambda values. A vertical line drawn at the lambda value corresponding to the minimum average cross-validation error. This line identifies the optimal level of model complexity, balancing bias, and variance to achieve the best predictive performance. (FIG. 4B, D) The partial likelihood deviation plotted against lambda using the LASSO model. These plots illustrate the trajectory of each predictor's coefficient as the regularization parameter (L1 Norm or lambda) changes, helping to identify which predictors are most influential in the model. Each line in the plot represents the coefficient of a predictor variable in the model, plotted against varying values of lambda. As lambda increases, the plot shows how each coefficient is shrunk towards zero. The entry or exit of lines across the zero line indicates when predictors are being added to or removed from the model highlighting their relative importance.

FIG. 5A-I. Validation of the identified gene signatures associated with platinum-paclitaxel using GEPIA2. Comparison of expression of (FIG. 5A) ICAM1 (FIG. 5B), TUBB2A (FIG. 5C), GLDC (FIG. 5D), PLAU, (FIG. 5E) AURKA, (FIG. 5F) NEAT1, (FIG. 5G) MXRA5, (FIG. 5H) GSN, and (FIG. 5I) MUC16 between ovarian cancer tissues and normal tissues. The red asterisk symbol above the boxplots indicates statistical significance between tumor and normal tissues. A single asterisk represents a p-value less than 0.05.

FIG. 6A-J. Validation of the identified gene signatures associated with platinum-only using GEPIA2. Comparison of expressions of (FIG. 6A) FCGBP (FIG. 6B), TFPI (FIG. 6C), NUAK1 (FIG. 6D), LRRC17, (FIG. 6E) FLRT2, (FIG. 6F) IL12A, (FIG. 6G) HSPA2, (FIG. 6H) CDC20, (FIG. 6I) FOXM1, and (FIG. 6J) MAP4K2 between ovarian cancer tissues and normal tissues. The red asterisk symbol above the boxplots indicates statistical significance between tumor and normal tissues. A single asterisk represents a p-value less than 0.05. The red dot represents an outlier, indicating that the expression level of a particular sample is much higher or lower than the rest of the data in the tumor group.

FIG. 7. Schematic representation of the signaling pathways for the gene signatures predicting in the response of serous ovarian cancer patients to platinum-paclitaxel. (Green color—under expression; red color—over expression; orange color—activation; dashed lines—indirect relationship; solid lines—direct relationship). Abbreviations: AURKA, Aurora Kinase A; AP-1, Activator Protein 1; GSN, Gelsolin; GLDC, Glycine Decarboxylase; ICAM1, Intercellular Adhesion Molecule 1; MXRA5, Matrix Remodeling Associated 5; MUC16, Mucin-16; NEAT1, Nuclear-Enriched Abundant Transcript 1; NPM1, Nucleophosmin 1; PLAU, Urokinase-Plasminogen Activator; TUBB2A, Tubulin Beta 2A; TP53, Tumor Protein 53.

FIG. 8. Schematic representation of the signaling pathways for the gene signatures predicting the response of serous ovarian cancer patients to platinum-only. (Green color—under expression; red color—over expression; dashed lines—indirect relationship; solid lines—direct relationship). Abbreviations: CDC20, Cell Division Cycle 20; HSPA2, Heat Shock Protein Family A (Hsp70) member 2; IL-12A, Interleukin 12 A; FCGBP, Fc Gamma Binding Protein; FOXM1, Forkhead box M1; FLRT2, Fibronectin leucine-rich transmembrane protein 2; LRRC17, Leucine-rich repeat containing 17; MAP4K2, MAPK Kinase Kinase Kinase 2; NUAK1, NUAK Family Kinase 1; TFPI, Tissue factor pathway inhibitor.

### DETAILED DESCRIPTION

Unless defined otherwise, all technical and scientific terms used herein have the same meaning as commonly understood to one of ordinary skill in the art to which this disclosure belongs. Although any methods, devices, and materials similar or equivalent to those described herein can be used in the practice or testing of the methods, devices and materials, the preferred methods, devices, and materials are now described.

### Definitions

The term “microarray” refers to an arrangement of locations on a device. The locations can be arranged in two-dimensional arrays, three-dimensional arrays, or other matrix formats. The number of locations may range from several to at least hundreds of thousands with each location representing a totally independent reaction site. A “nucleic acid array” refers to an array containing nucleic acid probes, such as oligonucleotides or larger portions of genes. The nucleic acid on the array may be single-stranded. As used herein, a nucleic acid or other molecule attached to an array is referred to as a “probe” or “capture probe.” Such probes include proteins, antibodies, small molecules that may bind to the differentially expressed gene products. Said probes may be linked to a detection molecule such as, for example, a fluorescent signal.

The term “biological sample,” as used herein, refers to a sample obtained from an organism or from components (e.g., cells) of an organism. The sample may be of any biological tissue or fluid. The sample may be a “clinical sample” which is a sample derived from a patient.

A nucleotide sequence is “complementary” to another nucleotide sequence if each of the bases of the two sequences match, that is, are capable of forming Watson-Crick base pairs. The term “complementary strand” is used herein interchangeably with the term “complement.” The complement of a nucleic acid strand may be the complement of a coding strand or the complement of a non-coding strand.

“Differential gene expression pattern” between, for example, a control cell and a test cell refer to a pattern reflecting the differences in gene expression between the control cell and the test cell. A differential gene expression pattern may also be obtained between a cell at one time point and a cell at another time point, or between a cell derived from a patient treated with a chemotherapeutic drug and a cell derived from the patient prior to drug treatment.

The term “expression profile” refers to a set of values representing mRNA levels of one or more genes in a cell. An expression profile may comprise, for example, values representing expression levels of at least about 2 genes, at least about 5 genes, at least about 10 genes, or at least about 50, 100, 200 or more genes.

The phrase “level of expression” refers to the level of mRNA, as well as pre-mRNA nascent transcript(s), transcript processing intermediates, mature mRNA(s), and degradation products, encoded by a gene in the cell. The phrase “level of expression” also refers to the level of protein or polypeptide in a cell.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a compound that is differentially present (i.e., increased or decreased) in a biological sample from a subject or a group of subjects having a first phenotype (e.g., responding to drug treatment) as compared to a biological sample from a subject or group of subjects having a second phenotype (e.g., not responding to a drug treatment). A biomarker may be differentially present at any level but generally should have an adjusted p-value (also known as FDR) or equal to or less than 0.05 and a Log2FC change of at least 1 (increase in expression) and below −1 (decrease in expression). Tables 6-7 represent the FDR and Log2FC levels of expression detected between responders versus non-responders.

As used herein, the terms “comparing” or “comparison” refers to making an assessment of how the level or expression of one or more biomarkers in a sample from a patient compares to levels of expression established for chemotherapy responder and/or non-responder samples.

As used herein, the terms “indicates” or “correlates” (or “indicating” or “correlating,” or “indication” or “correlation,” depending on the context) in reference to a parameter, e.g., the level of expression of a biomarker gene in a sample from a may mean that the patient is likely, or unlikely, to respond to chemotherapy. In specific embodiments, the parameter may comprise the level of expression of one or more biomarkers as disclosed herein.

The terms “measuring” and “determining” are used interchangeably throughout and refer to methods which include obtaining or providing a patient sample and/or detecting the level of biomarker expression in a sample. In certain embodiments, the terms are also used interchangeably with the term “quantitating.”

The present disclosure generally relates to gene expression profiling of tissue samples obtained from ovarian cancer patients who are candidates for chemotherapy treatment. Said samples are obtained from patients and levels of biomarker expression are compared to established responder versus non-responder levels of biomarker expression. More specifically, the present disclosure provides methods, based on characterization of gene expression, which allow a physician to predict whether a SOC patient is likely to respond well to treatment with a chemotherapeutic reagent.

In an embodiment, the chemotherapy reagents are platinum-paclitaxel. In yet another embodiment, the chemotherapy reagent is platinum only. Platinum agents include, for example, those reagents that covalently bind to DNA making DNA crosslinks and, eventually, inhibiting the cell cycle and cell proliferation. Such platinum reagents include, but are not limited to, cisplatin or carboplatin. Taxane agents are molecules capable of inducing cellular death by binding to tubulin and inhibiting the disassembly of microtubules required for chromosome segregation and cell division. In a non-limiting embodiment, the taxane agent is paclitaxel.

In one embodiment, the disclosure provides a method for predicting the likelihood that patients diagnosed with SOC, who are candidates for platinum-paclitaxel or platinum-only chemotherapy, will respond to such treatment, comprising determining the expression level of one or more biomarker transcripts, or their expression product, in a SOC patient sample, wherein the biomarker transcript is the transcript of one or more genes selected from the group consisting of: ICAM1, TUBB2A, GLDC, PLAU, AURKA, NEAT1, MXRA5, GSN, and MUC16 and/or FCGBP, TFPI, NUAK1, LRRC17, FLRT2, IL12A, HSPA2, CDC20, FOXM1, and MAP4K2.

In one embodiment, the disclosure provides a method for predicting the likelihood that patients diagnosed with SOC, who are candidates for platinum-paclitaxel chemotherapy, will respond to such treatment, comprising determining the expression level of one or more biomarker RNA transcripts, or their expression product, in a patient sample, wherein the transcript is the transcript of one or more genes selected from the group consisting of: ICAM1, TUBB2A, GLDC, PLAU, AURKA, NEAT1, MXRA5, GSN, and MUC16.

In one embodiment, the disclosure provides a method for predicting the likelihood that patients diagnosed with SOC, who are candidates for platinum-only chemotherapy, will respond to such treatment, comprising determining the expression level of one or more biomarker RNA transcripts, or their expression product, in a patient sample, wherein the biomarker transcript is the transcript of one or more genes selected from the group consisting of: FCGBP, TFPI, NUAK1, LRRC17, FLRT2, IL12A, HSPA2, CDC20, FOXM1, and MAP4K2.

In one aspect, the disclosure relates to a method for predicting the response of a SOC patient to platinum-paclitaxel chemotherapy comprising the steps of:
(i) determining in a sample isolated from said patient the expression levels of one or more biomarkers, or their expression product, wherein the biomarker is the transcript, or protein product, of one or more genes selected from the group consisting of: ICAM1, TUBB2A, GLDC, PLAU, AURKA, NEAT1, MXRA5, GSN, and MUC16; and
(ii) comparing the expression levels of said one or more genes obtained in step (i) with established expression levels for responders and/or non-responders wherein similarities in the established expression levels for either responders or non-responders is indicative of whether the patient will respond to platinum-paclitaxel chemotherapy. In one embodiment, the sample is derived from the SOC patient before drug treatment. In another embodiment, the sample is derived the SOC patient after initiation of drug treatment.

In one aspect, the disclosure relates to a method for predicting the response of a SOC patient to platinum-only chemotherapy comprising the steps of:
(i) determining in samples isolated from said patient the expression levels of one or more biomarkers, or their expression product, wherein the biomarker is the transcript, or protein product, of one or more genes selected from the group consisting of: FCGBP, TFPI, NUAK1, LRRC17, FLRT2, IL12A, HSPA2, CDC20, FOXM1, and MAP4K2; and
(ii) comparing the expression levels of said one or more genes obtained in step (i) with established expression levels for responders and/or non-responders wherein similarities in the established expression levels for either responders or non-responders is indicative of whether the patient will respond to platinum-only chemotherapy.

The present disclosure also provides a method of preparing a prognostic profile for a SOC patient using each of the disclosed methods above for predicting the response of a SOC patient to platinum-paclitaxel or platinum-only chemotherapy. Additionally, each of the disclosed methods above may further comprise the step of creating a report summarizing the data obtained by said gene expression analysis. In yet another embodiment, the disclosed methods above may further comprise the administration of platinum-paclitaxel or platinum-only chemotherapy where it is determined that the patient is likely to respond to such drug treatment.

Accordingly, in a further embodiment, where the method for predicting the responsiveness of an SOC patient to platinum-paclitaxel chemotherapy indicates a likelihood of responding to said chemotherapy, the chemotherapy is administered to the SOC patient. In a further embodiment, where the method for predicting the responsiveness of an SOC patient to platinum-only chemotherapy indicates a likelihood of responding to said chemotherapy, the chemotherapy is administered to the SOC patient.

The term “reference sample”, “control sample”, as used herein, relates to a sample, which contains reference nucleic acids or proteins to be used as a source of reference nucleic acids or proteins for the methods disclosed herein. In a preferred embodiment, the reference samples are sample derived from chemotherapy responders and/or non-responders. The biomarker nucleic acid or protein levels are then determined in said reference sample and the value obtained is then compared with the levels of the protein or nucleic acid in the patient test sample. This allows the designation of the test sample as “low,” “normal” or “high” expression. The collection of samples from which the reference level is derived will preferably be constituted from subjects suffering from the same type of cancer, i.e. SOC and undergoing either platinum-paclitaxel or platinum-only chemotherapy.

In an embodiment, a biomarker expression profile may be developed for a SOC patient, for determining their likelihood of responding to platinum-paclitaxel or platinum-only chemotherapy, based on the expression of the one or more biomarker genes disclosed herein wherein said expression includes both increases and decreases in specific biomarker expression associated with the likelihood of responding to chemotherapy.

In particular embodiments, the methods disclosed herein include collecting a biological sample, such as a primary ovarian tumor sample in which expression of a biomarker gene can be detected. Biological samples may be obtained from a subject by a variety of techniques including, for example, by scraping or swabbing an area, by using a needle to aspirate cells, or by removing a tissue sample (i.e., biopsy). Methods for collecting such biological samples are well known in the art. In some embodiments, a ovarian tumor sample is obtained by, for example, fine needle aspiration biopsy, core needle biopsy, or excisional biopsy. Fixative and staining solutions may be applied to the cells or tissues for preserving the specimen and for facilitating examination. Biological samples, particularly ovarian tumor samples, may be transferred to a glass slide for viewing under magnification. In one embodiment, the biological sample is a formalin-fixed, paraffin-embedded tissue sample, particularly a primary ovarian tumor sample.

The expression levels of the one or more biomarker mRNA transcripts, or their protein products, can be determined by methods known in the art. Methods for detecting expression of the biomarker genes disclosed herein, include methods based on hybridization analysis of polynucleotides, methods based on sequencing of polynucleotides, immunohistochemistry methods, and proteomics-based methods. The methods generally detect expression products (e.g., mRNA or protein) of the biomarker genes. In preferred embodiments, PCR-based methods, such as reverse transcription PCR (RT-PCR), and array-based methods are used.

Many expression detection methods are based on the use of isolated RNA. The starting material is typically total RNA isolated from a biological sample, such as a tumor or tumor cell line, and corresponding normal tissue or cell line, respectively. If the source of RNA is a primary tumor, RNA (e.g., mRNA) can be extracted, for example, from frozen or archived paraffin-embedded and fixed (e.g., formalin-fixed) tissue samples (e.g., pathologist-guided tissue core samples). General methods for RNA extraction are well known in the art and are disclosed in standard textbooks of molecular biology, including Ausubel et al., ed., Current Protocols in Molecular Biology, John Wiley & Sons, New York 1987-1999. The isolated RNA may be further processed for further purification or selection, e.g., selection for mRNA.

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the isolated RNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to a biomarker gene transcript, or any derivative DNA or RNA. Hybridization of the isolated RNA with the probe indicates that the biomarker gene in question is being expressed. In an embodiment, the nucleic acid probes are designed to hybridize to the biomarker gene transcripts disclosed herein.

In one embodiment, the RNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in a gene chip array. A skilled artisan can readily adapt known RNA detection methods for use in detecting the level of expression of the biomarker genes of the present disclosure.

Reagents for detecting the biomarker include one or more reagents for detecting the RNA expression level of the biomarker in the sample, or a reagent for detecting the protein expression level of the biomarker in the sample. Reagents for detecting the RNA expression level of the biomarker in the sample includes reagents used in methods that include, but are not limited to, PCR-based detection method, southern hybridization methods, northern hybridization method, dot hybridization method, fluorescence in situ hybridization method, DNA microarray methods, PCR-ASO probe method, high-throughput sequencing platform methods, and chip methods. In an embodiment, the reagent for detecting the biomarker comprises one or more of a primer for specifically amplifying the biomarker, a probe for specifically recognizing the biomarker, i.e., a nucleic acid probe, and/or a binding agent for specifically binding to a protein encoded by the biomarker.

In an embodiment a method is provided for determining a patient's prognosis for responding to platinum-paclitaxel or platinum-only chemotherapy comprising the steps of: (i) providing a nucleic acid probe comprising a nucleotide sequence having at least 10, at least 15, at least 25 or at least 40 consecutive nucleotides complementary to the one or more biomarkers disclosed herein, the expression levels of which correlate to a SOC patient's likelihood of responding to platinum-paclitaxel or platinum-only chemotherapy; (ii) contacting the nucleic acid probe under stringent conditions with the RNA of a patient's tissue sample; and (iii) detecting the amount of hybridization, wherein a difference in the amount of hybridization with the RNA of the patient's test sample as compared to the amount of hybridization of a control test sample is indicative of the patient's prognosis for responding to platinum-paclitaxel or platinum-only chemotherapy.

To compare expression levels, labeled nucleic acids may be contacted with the test sample under conditions sufficient for binding between the target sample nucleic acid and the probe. In one embodiment, the hybridization conditions may be selected to provide for the desired level of hybridization specificity; that is, conditions sufficient for hybridization to occur between the target sample nucleic acid and probes.

Hybridization may be carried out in conditions permitting essentially specific hybridization. The length and GC content of the nucleic acid will determine the thermal melting point and thus, the hybridization conditions necessary for obtaining specific hybridization of the probe to the target sample nucleic acid. These factors are well known to a person of skill in the art and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed. Elsevier, N.Y., (1993)).

In more particular embodiments, an assay performed on a biological sample obtained from a subject may comprise extracting nucleic acids from the biological sample. The assay can further comprise contacting nucleic acids with one or more primers that specifically bind one or more biomarkers described herein to form a primer: biomarker complex. The assay can further comprise the step of amplifying the primer: biomarker complexes. The amplified complexes can then be detected/quantified to determine a level of expression of the one or more biomarkers. A patient's likelihood of responding to platinum-paclitaxel or platinum-only chemotherapy can then be identified based on a comparison of the measured levels of one or more biomarkers described herein to one or more reference controls as described herein. The subject can then be treated appropriately, based on the observed levels of gene expression.

In particular aspects, biomarker gene expression is assessed by quantitative RT-PCR. Numerous different PCR or QPCR protocols are known in the art and can be directly applied or adapted for use using the presently described compositions for the detection and/or quantification of the biomarker gene transcripts disclosed herein. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. In certain embodiments, the biomarkers of the present disclosure can be measured by polymerase chain reaction (PCR).

In certain specific embodiments, the present disclosure contemplates quantitation of one or more biomarkers described herein for use in prognosing a SOC patient's response to platinum-paclitaxel or platinum-only chemotherapy. The one or more biomarkers can be quantitated, and the expression can be compared to reference levels. Overexpression or under expression, depending on the biomarker, relative to the reference is indicative of the likelihood of responding to platinum-paclitaxel or platinum-only chemotherapy. PCR can include quantitative type PCR, such as quantitative, real-time PCR.

In a specific embodiment, the quantitation steps are carried out using Quantitative PCR (QPCR) (also referred as real-time PCR). One of ordinary skill in the art can design primers that specifically bind and amplify one or more biomarkers described herein using the publicly available sequences thereof. QPCR is preferred under some circumstances because it provides not only a quantitative measurement, but also reduced time and contamination. QPCR gene measurement can be applied to standard formalin-fixed paraffin-embedded clinical tumor blocks, such as those used in archival tissue banks and routine surgical pathology specimens.

In order to normalize the values of mRNA expression among the different samples, it may be desirable to compare the expression levels of the mRNA of interest in the test samples with the expression of a control RNA which is an RNA whose expression levels do not change or change only in limited amounts in tumor cells with respect to non-tumorigenic cells. Such control RNAs may be derived from housekeeping genes and which code for proteins which are constitutively expressed and carry out essential cellular functions. Examples of housekeeping genes for use in the disclosed methods include β-2-microglobulin, ubiquitin, 18-S ribosomal protein, cyclophilin, GAPDH and β-actin.

In another embodiment, microarrays are used for expression profiling. DNA microarrays provide one method for the simultaneous measurement of the expression levels of large numbers of biomarkers. Each array consists of a reproducible pattern of capture probes attached to a solid support. Labeled RNA or DNA is hybridized to complementary probes on the array and then detected by detection of label. Hybridization intensities for each probe on the array are determined and converted to a quantitative value representing relative gene expression levels. High-density oligonucleotide arrays are particularly useful for determining the gene expression profile for a large number of RNAs in a sample.

By “microarray” is intended an ordered arrangement of hybridizable array elements, such as, for example, polynucleotide probes, on a substrate. The term “probe” refers to any molecule that is capable of selectively binding to a specifically intended target biomarker, for example, a nucleotide transcript or a protein encoded by or corresponding to a biomarker. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules. Arrays can be nucleic acids (or peptides) on beads, gels, polymeric surfaces, fibers (such as fiber optics), glass, or any other appropriate substrate. Arrays can be packaged in such a manner as to allow for diagnostics or other manipulation of an all-inclusive device.

In a specific embodiment of the microarray technique, PCR amplified inserts of cDNA clones are applied to a substrate in a dense array. The biomarker genes, immobilized on the microchip, are suitable for hybridization under stringent conditions. Fluorescently labeled cDNA probes can be generated through incorporation of fluorescent nucleotides by reverse transcription of RNA extracted from the ovarian tumor tissue of interest. Labeled cDNA probes applied to the chip hybridize with specificity to each spot of DNA on the array. After stringent washing to remove non-specifically bound probes, the chip is scanned for detection of label. The quantitation of hybridization of each arrayed element allows for assessment of corresponding mRNA abundance.

In a specific aspect, microarrays are provided comprising one or more probes corresponding to biomarker genes demonstrated to have altered expression following exposure to platinum-paclitaxel or platinum-only chemotherapy. In an embodiment, the microarray may be a microarray comprising one or more of the genes disclosed herein the expression levels of which correlate to a SOC patient's likelihood of responding to platinum-paclitaxel or platinum-only chemotherapy. In another aspect, methods are provided for using said microarrays to provide a patient's prognosis for responding to platinum-paclitaxel or platinum-only chemotherapy. The microarray may comprise, for example, probes corresponding to at least 2, at least 5, at least 10, at least 100 biomarker genes characteristic of the expression levels of which correlate to platinum-paclitaxel or platinum-only chemotherapy. The microarray may comprise probes corresponding to each biomarker gene or gene product disclosed herein.

In an embodiment, the microarray may be a microarray comprising one or more of the genes disclosed herein the expression levels of which correlate to a SOC patient's likelihood of responding to platinum-paclitaxel chemotherapy selected from the group consisting of: ICAM1, TUBB2A, GLDC, PLAU, AURKA, NEAT1, MXRA5, GSN, and MUC16.

In an embodiment, the microarray may be a microarray comprising one or more of the genes disclosed herein the expression levels of which correlate to a SOC patient's likelihood of responding to platinum only chemotherapy selected from the group consisting of: FCGBP, TFPI, NUAK1, LRRC17, FLRT2, IL12A, HSPA2, CDC20, FOXM1, and MAP4K2.

The methods described above result in the production of hybridization patterns of labeled target nucleic acids on the array surface. The resultant hybridization patterns of labeled nucleic acids may be visualized or detected in a variety of ways, with the particular manner of detection selected based on the label of the target nucleic acid. Representative detection means include scintillation counting, autoradiography, fluorescence measurement, colorimetric measurement, light emission measurement, light scattering, and the like.

Any conventional method can be used within the context of the present disclosure to quantify the levels of biomarker protein. For example, biomarkers can be detected and/or measured by immunoassays, mass spectroscopy, western blots and other proteomic detection methods known to one of skill in the art. By way of non-limiting example, the levels of said proteins can be quantified by means of conventional methods, for example, using antibodies with a capacity to specifically bind to biomarker protein (or to fragments thereof containing antigenic determinants) and subsequent quantification of the resulting antibody-antigen complexes.

Such immunoassays require bio-specific capture reagents/binding agent, such as antibodies, to capture the biomarkers. Many antibodies are available commercially. The present disclosure contemplates traditional immunoassays including, for example, sandwich immunoassays including ELISA or fluorescence-based immunoassays, immunoblots, Western Blots (WB), as well as other enzyme immunoassays. Binding of the antigen to the antibody results in changes in absorbance, which is measured.

In specific embodiments, the levels of expression of the biomarkers are determined by contacting the biological sample with antibodies, or antigen binding fragments thereof, that selectively bind to the biomarkers; and detecting binding of the antibodies, or antigen binding fragments thereof, to the biomarkers. In certain embodiments, the binding agents employed in the disclosed methods and compositions are labeled with a detectable moiety. The detection can be performed using a second antibody to bind to the capture antibody complexed with its target biomarker.

The antibodies to be employed in these assays can be, for example, polyclonal sera, hybridoma supernatants or monoclonal antibodies, antibody fragments, Fv, Fab, Fab′y F(ab′)2, ScFv, diabodies, triabodies, tetrabodies and humanised antibodies. The antibodies can be labeled or not. Examples of labels which can be used include radioactive isotopes, enzymes, fluorophores, chemiluminescent reagents, enzymatic substrates or cofactors, enzymatic inhibitors, particles, colorants, etc. There are a wide variety of well-known assays that can be used, which use non-labeled antibodies (primary antibody) and labeled antibodies (secondary antibodies); among these techniques are included Western-blot or Western transfer, ELISA (enzyme linked immunosorbent assay), RIA (radioimmunoassay), competitive EIA (enzymatic immunoassay), DAS-ELISA (double antibody sandwich ELISA), immunocytochemical and immunohistochemical techniques, techniques based on the use of biochips or protein microarrays.

The present disclosure also provides kits which are suitable for the determination of the expression levels of the biomarker genes disclosed herein. These kits are useful for analyzing a sample from a patient suffering from SOC and to design personalized therapies for said patients based on the results obtained. In a particular embodiment, the reagents of the kit are capable of specifically detecting the levels of the mRNA encoded by a biomarker gene as disclosed above. In another embodiment, the reagents of the kit are capable of specifically detecting the levels of a biomarker protein as disclosed above. The kits may be designed for use with a specific type of SOC patient, e.g., any stage of SOC, early stage of SOC or metastatic SOC. Such kits may be in a microarray format and interface with data analysis operations disclosed below and may be analyzed using a computing system as disclosed below.

In a specific embodiment a kit is designed for predicting the likelihood that a patient diagnosed with SOC, who are candidates for treatment with platinum-paclitaxel chemotherapy, will respond to such treatment, wherein said kit is designed to determine the expression level of one or more biomarker transcripts, or their expression product, in a SOC patient sample, wherein the biomarker transcript is the transcript of one or more genes selected from the group consisting of: ICAM1, TUBB2A, GLDC, PLAU, AURKA, NEAT1, MXRA5, GSN, and MUC16.

In one embodiment, a kit is designed for predicting the likelihood that patients at early stages of SOC, who are candidates for treatment with platinum-only chemotherapy will respond to such treatment, wherein said kit is designed to determine the expression level of one or more biomarker RNA transcripts, or their expression product, in a SOC patient sample, wherein the biomarker transcript is the transcript of one or more genes selected from the group consisting of: FCGBP, TFPI, NUAK1, LRRC17, FLRT2, IL12A, HSPA2, CDC20, FOXM1, and MAP4K2.

In certain embodiments, the kit may further include instructions for using the kit, solutions for suspending or fixing cells derived from the sample, detectable tags or labels and solutions for lysing cells. In some instances, the kit may contain solutions and reagents for detecting the RNA or protein products of the biomarker genes.

A computing system may be used to identify genes that may be predictive of whether a person having serous ovarian cancer would be responsive or non-responsive to chemotherapy. The computing system may be any system capable of performing computations, including, but not limited to, a desktop, a laptop, a server, a smartphone, a smart watch, a tablet, a wearable device, a cloud system, a standalone system, or other type of computing system, or may be any circuit capable of performing computations, including, but not limited to, an application specific integrated circuit (ASIC), a microprocessor, a microcontroller, a central processing unit (CPU), a graphics processing unit (GPU), a field-programmable gate array, and/or a programmable logic device, among other circuits. In various embodiments, a computing system may include one or more processors and one or more memory storing instructions which, when executed by the one or more processors, implement one or more of the computations and/or procedures disclosed in the present disclosure.

Data for identifying genes that may be predictive may be gathered and pre-processed by a computing system in various ways. For example, data which may be an outlier, as compared to previously known data or reference data, may be identified as an outlier and may not be further processed. As another example, various data processing may be employed, such as, without limitation, corrections, transformations, and/or normalizations. Examples of data corrections, transformation, and normalizations are described below herein. Such examples are merely illustrative and do not limit the scope of the present disclosure.

In accordance with aspects of the present disclosure, the gathered data may be used by a computing system to compute a prediction score. Various prediction scores are disclosed below herein. The prediction scores may be compared to one or more threshold values to determine a predicted outcome. For example, if a prediction score has a value greater than a threshold value, the predicted outcome may be that a person would be responsive to chemotherapy, and if the prediction score has a value less than a threshold value, the predicted outcome may be that the person would non-responsive to chemotherapy. These predicted outcomes are merely an example, and other predicted outcomes are contemplated to be within the scope of the present disclosure.

In accordance with aspects of the present disclosure, a computing system may implement a trained machine learning model, and the trained machine learning model may process the gathered data to infer whether the gathered data reflects a predicted outcome. Various machine learning models, and various computations supporting such machine learning models, are disclosed below herein. Persons skilled in the art will understand how to implement and use such machine learning models and computations. In various embodiments, the machine learning model may be a classifier that classifies whether the gathered data is reflective of responsiveness to chemotherapy or is reflective of non-responsiveness to chemotherapy. In various embodiments, the machine learning model may be a regression model that provides an output value reflective of degree of responsiveness and/or non-responsiveness to chemotherapy. The output value may, for example, be compared to one or more threshold values to determine the predicted outcome. The machine learning models disclosed herein are merely examples, and other machine learning models are contemplated to be within the scope of the present disclosure.

In one embodiment, a system may be utilized that comprises a processing function that identifies specific patterns, for example, patterns relating to differential gene expression, for example, between the expression profile of a responder SOC tissue sample and the expression profile of a counterpart non-responder SOC tissue sample. The system may identify patterns of gene expression between more than two samples. Various algorithms are available for analyzing gene expression profile data, for example, the type of comparisons to perform, such as the algorithms disclosed below herein.

Comparison of the expression levels of one or more biomarkers characteristic of platinum-paclitaxel or platinum-only chemotherapy efficacy with reference expression levels, for example, expression levels in cells of responder SOC patients or in normal counterpart cells, may be conducted using computing systems. In one embodiment, expression levels may be obtained from two different tissue samples and the two sets of expression levels may be introduced into a computing system for comparison. For example, one set of expression levels is entered into a computing system for comparison with values that are already present in the computing system, or in computer-readable form that is then entered into the computing system.

In one embodiment, the computing system may also contain a database comprising values representing levels of expression of one or more biomarkers characteristic of platinum-paclitaxel or platinum-only chemotherapy efficacy. The database may contain one or more expression profiles of genes characteristic of small molecule efficacy in different cells.

The present disclosure also provides a machine-readable, processor-readable, or computer-readable medium including program instructions for performing the following steps: (i) comparing a plurality of values corresponding to expression levels of one or more biomarkers characteristic of platinum-paclitaxel or platinum-only chemotherapy efficacy in a test sample with a database including records comprising reference expression or expression profile data of one or more reference samples and an annotation of the type of sample; and (ii) indicating to which sample the test sample cell is most similar based on similarities of expression profiles. The reference cells may also be cells from subjects responding or not responding to platinum-paclitaxel or platinum-only chemotherapy.

The skilled person will not have problems in selecting a suitable statistical method to evaluate the biomarker marker combinations as disclosed herein and thereby obtain a suitable mathematical algorithm. In this embodiment, data obtained from analysis of biomarker gene expression is evaluated using one or more pattern recognition algorithms.

Unless otherwise defined, all technical and scientific terms used herein have the same meaning as commonly understood by one skilled in the art. Although methods and materials similar to or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLE

### 1. Materials and Methods

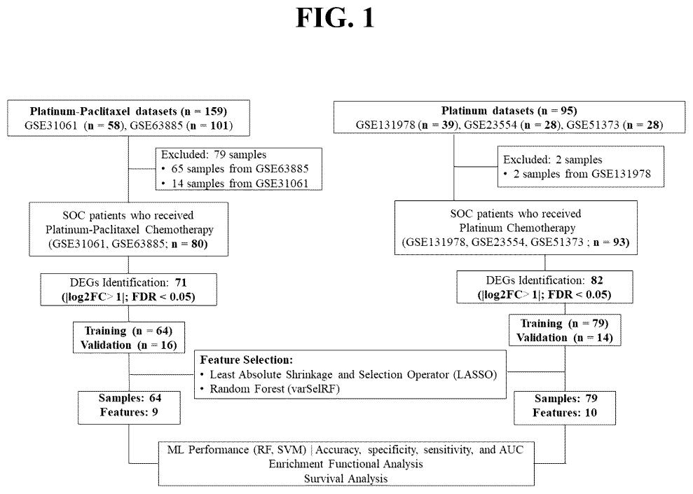

The proposed machine learning framework consists of five main steps: data cleaning and pre-processing, feature extraction, feature selection, classification using machine learning classifiers, and biological significance analysis as shown in FIG. 1.

### 1.1 Datasets

The set of binary files in a CEL format containing the ovarian cancer raw gene expression data were retrieved from the Gene Expression Omnibus (GEO) database using “GEOquery” R/Bioconductor library (www.ncbi.nlm.nih.gov/geo/). The terms “Serous-Ovarian-Cancer”, “Chemotherapy”, “Expression profiling by array”, and “Homo-sapiens” were used to find relevant experimental studies that examined the gene expression profiles of ovarian tumors in patients who either responded or did not respond to the drug. The chemotherapy regimens of interest are platinum-based chemotherapy. This methodology led to the identification of five datasets, including GSE131978, GSE23554, GSE51373, GSE63885, and GSE30161.

Clinicopathological information from the original studies was used for the analysis. The GSE131978 dataset contained samples from two different platforms, Affymetrix GeneChip Human Genome U133 Array (HG-U133A) and Affymetrix GeneChip Human Genome U133 Plus 2.0 Array (HG-U133Plus2). The samples were grouped based on the platform and were used as two separate datasets. The GSE131978-HG-U133A dataset contains 25 samples including 11 responders, 12 non-responders, and 2 samples with missing information. The two samples were removed from the dataset. The GSE131978-HG-U133Plus2 dataset, on the other hand, contains 14 samples including 7 responders and 7 non-responders. The GSE23554 dataset contains a total of 28 samples including 18 responders and 10 non-responders. The GSE51373 dataset contains a total of 28 samples including 16 responders and 12 non-responders. The GSE63885 dataset contains a total of 101 samples including 6 5 responders and 10 non-responders. However, only 36 samples (24 responders and 12 non-responders) were used for this analysis. The remaining 65 samples were excluded from the study because these samples were not extracted from a serous ovarian tissue and/or received a different chemotherapy regimen. The GSE30161 dataset contains a total of 58 samples including 54 responders, 1 non-responder, and 3 samples with missing information. Only 44 samples (25 responders and 19 non-responders) were included in this study. The remaining samples were excluded because they were not extracted from serous ovarian cancer tissues. The samples were divided and organized into two groups based on the type of chemotherapy administered. The ratio of responders to non-responders was biased in some of the deposited GEO datasets including GSE63885 and GSE30161. Thus, the datasets for each chemotherapy regimen were combined for better outcomes.

Table 1 displays the GEO accession numbers of the expression datasets, along with the corresponding platform used for each dataset. In addition, the table includes the number of samples categorized as responders, non-responders, and the total number of samples. The reference manuscripts for each dataset utilized in this study are specified as well.

TABLE 1
Description of each dataset for two different chemotherapy regimens.

| GEO | | | |
|---|---|---|---|
| Accession | Platform | Number of Samples | Title/Description |
| GSE131978 | GPL96 ¹ | 25 (11 responders + | FXYD5 (dysadherin) upregulation predicts |
| | | 12 non-responders + | shorter survival and reveals platinum resistance |
| | | 2 NA) | in high-grade serous ovarian cancer |
| | GPL570 ² | 14 (7 responders + | |
| | | 7 non-responders) | |
| GSE23554 | GPL96 ¹ | 28 (18 responders + | Ovarian Cancer Dataset |
| | | 10 non-responders) | |
| GSE51373 | GPL570 ² | 28 (16 responders + | Gene expression data from high grade serous |
| | | 12 non-responders) | ovarian cancer |
| GSE63885 | GPL570 ² | 101 (24 responders + | Gene expression profiling in ovarian cancer |
| | | 12 non-responders + | |
| | | 65 NA) | |
| GSE30161 | GPL570 ² | 58 (25 responders + | Genomic Multivariate Predictors of Response |
| | | 19 non-responders + | to Adjuvant Chemotherapy in Ovarian |
| | | 14 NAs) | Carcinoma: Predicting Platinum Resistance |

¹GPL96 = Affymetrix GeneChip Human Genome U133 Array (HG-U133A),

### ²GPL570=Affymetrix GeneChip Human Genome U133 Plus 2.0 Array (HG-U133Plus2).

### 1.2. Inclusion and Exclusion Criteria

The following criteria determine the patients' samples included in the study: (1) patients with serous ovarian cancer; (2) patients who underwent platinum-based chemotherapy; (3) a sample size of at least 10 for each dataset; (4) gene expression profiling datasets; and (5) available information about the drug response and/or recurrence and/or survival status. The exclusion criteria comprise: (1) datasets containing cell-line or xenograft samples; (2) samples with missing information about the drug type; and (3) samples with missing information about the drug response.

### 1.3. Machine Learning Framework

The machine learning framework followed methods to determine chemotherapy responders and non-responders from previously published work [25].

### 1.3.1 Data Pre-Processing, Quality Control, and Feature Extraction

The raw microarray expression data were retrieved from each GEO database. Certain samples were excluded from the raw non-normalized data because they contain missing information needed for the analysis. The Affymetrix data were analyzed using Guanine Cytosine Robust Multi-Array Analysis (GCRMA) from the Bioconductor package germa (version 2.44.0) [26] for the HG-U133A and HG-U133 Plus 2 platform types. The GCRMA algorithm conducts several data processing steps such as background correction, log2 transformation, quantile normalization, and summarization of probe sets into gene-level expression values [27,28]. The “nsFilter” function from the Bioconductor package genefilter (version 1.60.0) removed probes with minor sample variance and low median expression levels in the normalized dataset [29].

Quality control (QC) for each normalized dataset utilized the outlier removal strategy. The ArrayQualityMetrics R package (version 4.1.0) [30] was used for assurance and quality control of the microarray experiments. This approach enhances the effectiveness of meta-analysis and increases the ability to detect differentially expressed genes [31]. In the quality control procedure, samples that were identified as outliers were excluded from the relevant datasets. Following this, the raw data, which were devoid of any outliers, underwent a new round of normalization using the method elucidated in the preceding section. The normalized datasets were then used in further analysis.

All probes were mapped to their corresponding gene symbols, which serve as a universal identifier across platforms. The Official HUGO Gene Nomenclature Committee (HGNC) was employed as it is subject to rigorous curation and has been shown to improve the precision of scientific and public communication [32,33]. When there are multiple probes for a given gene symbol, the average expression value of all those probes was used to determine the expression level of that gene. Unannotated probes were disregarded from the analysis. The conversion process from probes to gene symbols was carried out using the R/Bioconductor package “org.Hs.eg.db” (version 3.14) [34]. Depending on the platform, the datasets were annotated with the R/Bioconductor packages hgu 133a.db or hgu133plus2.db.

### 1.3.2 Z-Score Transformation

Application of the Z-score transformation normalized the gene expression data using the “scale” function in R/Bioconductor package stats. This approach of normalization allows for consistent data across studies, allowing for direct comparison of microarray data regardless of differences in the initial hybridization intensities [35]. This approach has been used extensively in previous studies and has consistently shown effective performance in many applications [36].

### 1.3.3 Batch Effect Correction

The issue of batch effects, which are systematic non-biological differences that can occur in multi-batch datasets due to variations in experimental conditions, was addressed in this study. To correct for batch effects and ensure the consistency of different batches, the ComBat algorithm from the ‘sva’ package in R was applied [37]. This method used an empirical Bayes framework to adjust for both known and unknown batch effects, thus normalizing the data. The application of ComBat enables us to mitigate potential confounders, thereby improving the reliability and comparability of the findings. This step was crucial for the following analysis, ensuring that the biological interpretations derived from the data were not obscured by technical variability.

### 1.3.4. Train/Test Split Using K-Fold Cross-Validation

To assess the machine learning model performance, the model first undergoes initial training using the training dataset, followed by an evaluation process on a validation set. The utilization of a cross-validation procedure is commonly used in situations where the dataset is constrained [38]. This process involves the iterative dividing of the data into distinct training and validation sets, which are subsequently used to train and assess the model, respectively. In this study, the training set split into 10 folds of approximately same size. An independent test dataset, on the other hand, refers to a distinct and independent set that has not been used in any capacity during the training and validation sets phases of the model. The R/Bioconductor caret package was used to randomly divide the samples into training and test sets using the function “create folds”.

### 1.4 Differential Expressed Genes (DEGs)

Differential gene expression analysis used the ‘limma’ package in R, which was specifically designed for the analysis of gene expression data derived from microarray [40]. The ‘limma’ approach uses a linear model framework coupled with empirical Bayes methods to provide robust statistical inference even when dealing with complex experiments and relatively small sample sizes. After pre-processing and normalization of the data, application of ‘limma’ allows the identify genes that demonstrated statistically significant changes in expression between responders and non-responders. Visualization of differentially expressed genes with the Enhanced Volcano plot allows the effective display of the statistical significance against the fold change on a log scale, highlighting genes that are biologically interesting and potentially warrant further study. This visual representation was important in facilitating a clearer understanding of the key results from the differential expression analysis.

### 1.5. Feature Selection

In this study, the Variable Selection Random Forest (varSelRF) and Least Absolute Shrinkage and Selection Operator (LASSO) methods were combined to extract genes with the best predictive power. The capacity of these methods to concentrate on a small set of genes with strong prediction power led to their selection. Furthermore, they necessitate minimal parameter tuning, as the default settings frequently result in optimal performance.

### 1.5.1. Least Absolute Shrinkage and Selection Operator (LASSO)

The LASSO technique is a form of regularization regression that is commonly used for the purpose of fitting a generalized linear model. The regression model is subjected to a penalty, specifically the LI norm, which leads to the decrease in regression coefficients for variables that make minimal contributions towards zero. LASSO regression analysis was applied using the R/Bioconductor package glmnet (version 4.1) [41]. The LASSO method exhibits strong performance in situations where the dataset demonstrates a high number of dimensions and a low sample size. Numerous studies have consistently shown that this method exhibits significant potential as a promising model for feature selection [42,43].

The acquired results and the regression coefficients were used to establish a scoring system that attributes weights to the chosen signatures. The formula employed is as follows:

$$Prediction Score = {\sum }_{i = 1}^{n} ( β i \times x i )$$

The provided formula uses the variables “n” to represent the number of genes in the gene signature and “B” is utilized to denote the regression coefficient associated with the chosen gene signatures. The regression coefficient is obtained through LASSO logistic regression. Furthermore, the symbol “x” represents the expression value that corresponds to the selected signature.

1.5.2. Variable Selection Random Forest (varSelRF)

The varSelRF method employs regression trees within the framework of random forest for the purpose of classification. The construction of the classification tree entails employing bootstrap samples, wherein each branch of the tree comprises a unique selection of candidate variables that are chosen randomly. The trees in varSelRF are generated using a method that integrates bootstrap aggregation (bagging) with feature selection within the random forest framework. Independent tree construction precedes the use of bagging and random variable selection methods to minimize inter-tree correlation, guaranteeing low-bias trees. The ntree parameter, representing the number of trees, was set to its default value of 2000, as was the mtry parameter, which determines the number of variables considered at each split [44].

### 1.6. Machine Learning Algorithms Performance

Two machine learning algorithms were used in this study: random forest and support vector machine. Random forest was applied via the R/Bioconductor package Random-Forest [45], while support vector machine was applied using the R/Bioconductor package e1071 [46]. Accuracy, sensitivity, and specificity were used as metrics to compare the efficacy of the models. To mitigate the risk of overfitting and enhance the robustness of the model evaluation, model tuning was implemented using the ‘caret’ package in R. This involved a systematic approach known as grid tuning in R, which allows for an extensive search over a predefined space of hyperparameter values to identify the most robust model sittings. For random forest, focus was on optimizing the ‘mtry’, which dictates the number of variables randomly sampled as candidates at each split, and the ‘ntree’, which represents the number of trees grown. Specifically, a tuning grid with varying levels of ‘mtry’ chosen based on the number of predictors, and a fixed ‘ntree’ value to evaluate the impact of these parameters on model complexity and accuracy. For SVM, the approach was to refine the cost of constraints violation ‘C’ and kernel width parameters ‘sigma’, using a preset range of values to determine the optimal balance between model simplicity and error minimization. The ‘trainControl’ function facilitated 10-fold-cross-validation, ensuring that the chosen hyperparameters provided robust predictions across different subsets of data. This systematic tuning via ‘caret’ package not only helped in identifying the most effective model settings, but also significantly contributed to the reliability and validity of the predictive models, thus achieving a balance between complexity and generalization.

All computational methods and figure generation were implemented using R language programming version 4.0.1. on an Intel Core-i9 CPU with 16 GB of RAM, and 64-bit Windows 10 configuration. Computations for machine learning could be run in approximately 1 h.

### 1.7. Biological Pathway Analysis

The canonical pathway enriched by differential genes was performed using Ingenuity Pathway Analysis (IPA), a web-based software application (Ingenuity Systems www.ingenuity.com accessed on 6 Jun. 2023) that identifies biological pathways and functions relevant to biomolecules of interest [47]. A core analysis was first constructed, and then a list of differential genes with their probe identification, FDR value and logarithmic fold change were uploaded to IPA [47]. Enrichment pathways of differential genes were generated based on the Ingenuity Pathway Knowledge Data Base.

### 1.8. Validation of the Expression Analysis

In this study, the Gene Expression Profiling Interactive Analysis (GEPIA2) online tool, accessible at www.gepia2.cancer-pku.cn/(accessed on 6 Jun. 2023) was used to conduct an analysis on the relevance of gene signatures in association with overall survival (OS) of patients diagnosed with ovarian cancer. The survival curve in the survival analysis was derived using the Kaplan-Meier method using the online tool accessible at www.kmplot.com/analysis/(accessed on 6 Jun. 2023). The log-rank test was used to assess the statistical significance of the observed difference. Statistical significance was determined by assessing the p-value, which was deemed significant if it was less than or equal to 0.05. Additionally, the median was employed as a cut-off criterion. Survival curves were generated incorporating hazard ratios (HR) and log-rank p-values for analysis.

### 2. Results

### 2.1. Data Extraction and Bactch Effect Analysis

Responder (49) and non-responder (31) samples meeting the inclusion criteria from the Affymetrix GeneChip human Genome U133 Plus 2.0 arrays platform (HG-U133 Plus 2) with a 54, 676 probes from the two GEO datasets, GSE301061 and GSE15888 were used for the platinum-paclitaxel analysis. The 54,676 hybridized probes utilized in these datasets targeted a total of 20,864 unique gene symbols, out of which the two datasets shared 15,167 gene symbols.

The same type of analyses was performed on datasets that contain gene expression profiles of SOC patients who received platinum-only. Responders (52) and non-responders (41) samples from the Affymetrix GeneChip human Genome U133 Plus 2.0 arrays platform (HG-U133_Plus_2) with a total of 54,676 probes as well as samples from the Affymetrix GeneChip human Genome U133a with 22,284 probes, were selected from the GEO datasets GSE31978 and GSE23554. Among these datasets, there were 6261 shared gene symbols.

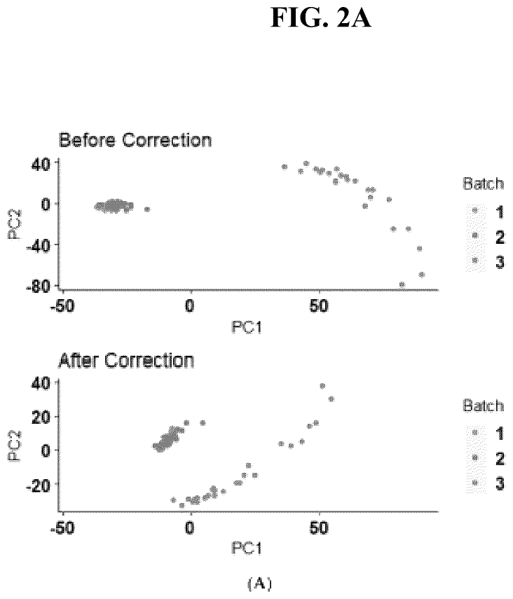

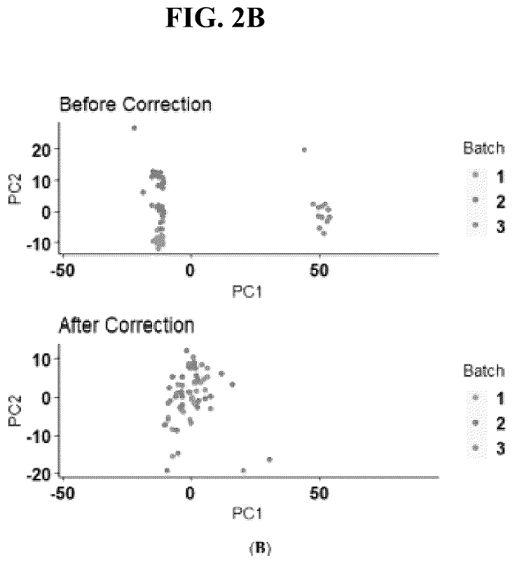

Interpretation of the batch effects and their correction is crucial for assessing the reliability of biological conclusions drawn from multi-batch datasets. The principal component analysis (PCA) illustrated in FIG. 2A-B demonstrates the distribution of data samples before and after a batch correction method application. Initially, the samples, as represented by the “Before” group, show a clear clustering pattern that likely reflects batch effects rather than underlying biological or experimental conditions (FIG. 2A-B). After the application of the batch correction technique, as seen in the “After” group, these clusters appear to have converged, indicating a reduction in batch effects (FIG. 2A-B). This reorganization not only highlights the effectiveness of the ComBat correction from the ‘sva’ package but also reinstates confidence in the biological insights derived from the data. The reduction in batch-associated variance and the enhanced alignment of clusters along biological variables highlights the robustness of the analytical approach, confirming that the observed differences in gene expression are due to underlying biological effects rather than technical artifacts. This correction essentially allows us to proceed with downstream analyses and interpretations with a higher degree of reliability, focusing on biological variations that are truly pertinent to the study.

Following the pre-processing steps, the merged dataset was split into training and validation sets using a ten-fold cross-validation approach. The training set of platinum-paclitaxel consisted of 64 samples, comprising 39 responders and 25 non-responders. The validation set, on the other hand, included 16 samples, with 10 responders and 6 non-responders. On the other hand, the training dataset for platinum-only comprised 74 samples, with 42 samples classified as responders and 32 samples classified as non-responders. The validation set comprised a total of 18 samples, consisting of 9 responders and 9 non-responders (FIG. 1).

2.2. Differential Expressed Genes Identified from the Platinum-Paclitaxel and Platinum-Only Data

Differentially expressed genes (DEGs) between the samples of responders and non-responders of the training set were determined using the “limma” package in R. The p-values were adjusted using the Benjamini-Hochberg (BH) method to control the false discovery rate (FDR), and a cut-off threshold of an adjusted p-value<0.05 was applied.

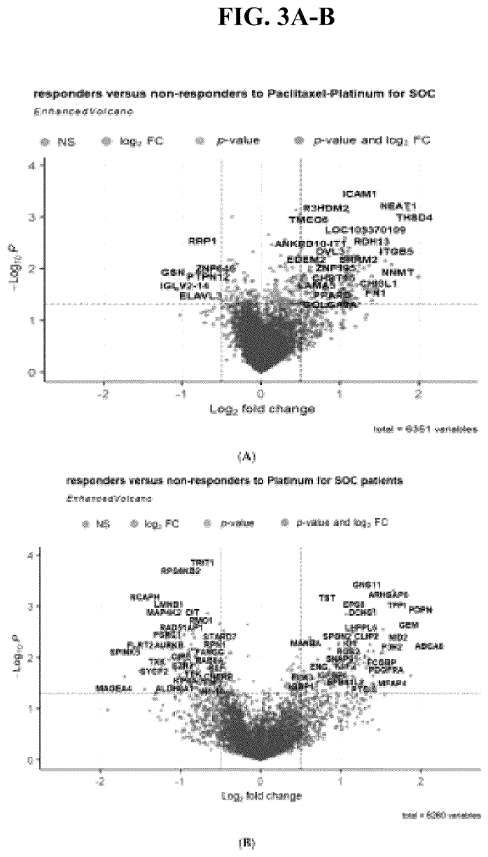

In total, 71 DEGs were identified between the tissue samples of non-responders and responders in patients with SOC who received platinum-paclitaxel treatment (Table 6). Among these DEGs, 69 genes were found to be upregulated, while only 2 genes were downregulated (FIG. 3A). However, 82 DEGs were identified when comparing the tissue samples of non-responders and responders in patients diagnosed with SOC who underwent platinum treatment (Table 7). Within the set of DEGs, it was observed that 58 genes were found to be upregulated, whereas 25 genes were downregulated (FIG. 3B).

2.3 Gene Signatures Identified from LASSO and varSeIRF Feature Selection Methods

To filter out the feature genes based on OC-related DEGs, two feature selection methods, LASSO regression and varSeIRF, were used.

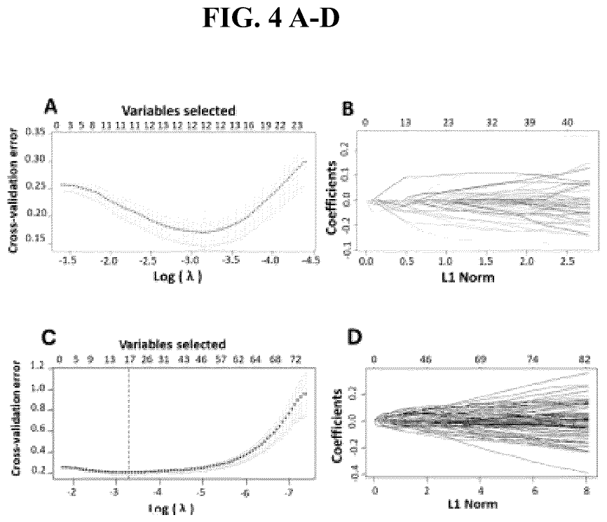

For LASSO analysis, ten-fold cross-validations were performed to calculate the cross-validation error and to determine the optimal lambda (2) value. The λ value corresponding to the minimum cross-validation error, denoted as λ min, was selected as the optimal λ. A dotted vertical line was plotted at the λ value chosen through 10-fold cross-validation. Out of 71 DEGs identified from the training set of platinum-paclitaxel, 12 non-zero coefficients (genes) were associated with minimum cross-validation error of 0.038 (FIG. 4A-B). These 12 genes are: ICAM1, TUBB2A, GLDC, PLAU, AURKA, SRRM2, DCHS1, NEAT1, MXRA5, NRBP2, GSN, and MUC16. The prediction score is computed using Equation (1).

Following the same concept on the platinum-only training data, out of 82-identified DEGs, 17 non-zero coefficients (genes) were associated with minimum cross-validation error of 0.0371 (FIG. 4C-D). The 17 gene signatures include NBL1, FCGBP, LMNB1, FLRT2, NUAK1, MAP4K2, SPINK5, LRRC17, SYCBP, TXK, IL 12A, CCN2, CORO2B, CLIP2, HSPA2, PAQR4, and TFPI. The prediction score is computed using Equation (1). As mentioned in the methodology section, the varSelRF method was used to confirm the results achieved by LASSO methods.

For the platinum-paclitaxel data, eleven feature genes were selected with the varSelRF method including, AEBP1, GSN, ICAM1, NEAT1, TUBB2A, PLAU, MXRA5, MUC16, GLDC, AURKA, and CD81. Nine feature genes were defined by overlapping the genes derived from these two feature selection methods, including Intercellular Adhesion Molecule 1 (ICAM1), Tubulin Beta 2A Class (TUBB2A), Glycine Decarboxylase (GLDC), Plasminogen Activator, Urokinase (PLAU), Aurora Kinase (AURKA), Nuclear-Enriched Abundant Transcript 1 (NEAT1), Matrix Remodeling Associated 5 (MXRA5), Gelsolin (GSN), and Mucin-16 (MUC16).

For the platinum-only training data, 13 feature genes were selected with the varSelRF method including LMOD1, FCGBP, TFPI, NUAK1, SPINK5, LRRC17, FLRT2, CCN2, IL12A, HSPA2, CDC20, MAP4K2, and FOXM1. A total of ten feature genes were defined by overlapping the genes derived from these two feature selection methods, including Fc Gamma Binding Protein (FCGBP), Tissue factor pathway inhibitor (TFPI), NUAK Family Kinase 1 (NUAK1), Cell Division Cycle 20 (CDC20), Leucine-Rich Repeat Containing 17 (LRRC17), Fibronectin Leucine-Rich Transmembrane Protein 2 (FLRT2), CCN2, Interleukin 12A (IL 12A), Heat Shock Protein Family A (Hsp70) Member 2 (HSPA2), Forkhead Box M1 (FOXM1), and mitogen-activated protein kinase kinase kinase kinase 2 (MAP4K2).

### 2.4. Validation of the Gene Signatures Using the GEPIA Database

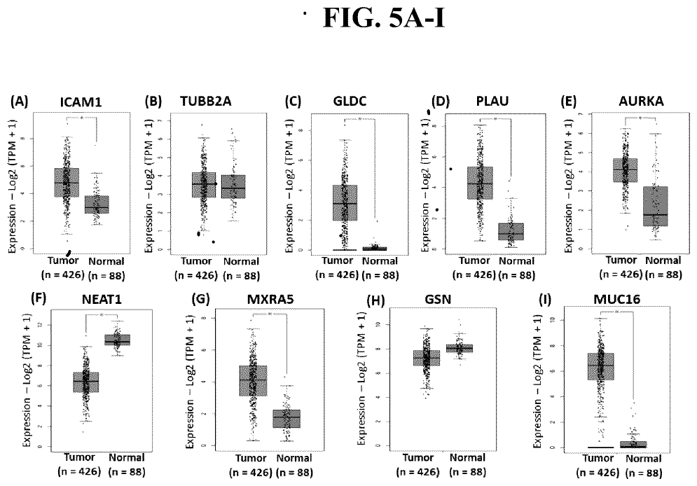

The mRNA expression of the gene signatures in normal and OC tissues was analyzed using GEPIA2 software accessible at www. gepia2.cancer-pku.cn/to validate the expression of the identified gene signatures. Based on datasets from databases such as TCGA and GTEx, the results reveal that the mRNA levels of ICAM1, GLDC, PLAU, AURKA, MXRA5, and MUC16 were significantly higher in OVCA than in normal tissues (FIG. 5A,B-E,G,I). In contrast, the mRNA level of NEAT1 was significantly lower in OVCA than in normal tissues (FIG. 5F). There was no statistically significant difference in the expression levels of TUBB2A and GSN between tumor and normal tissues (FIG. 5B, H).

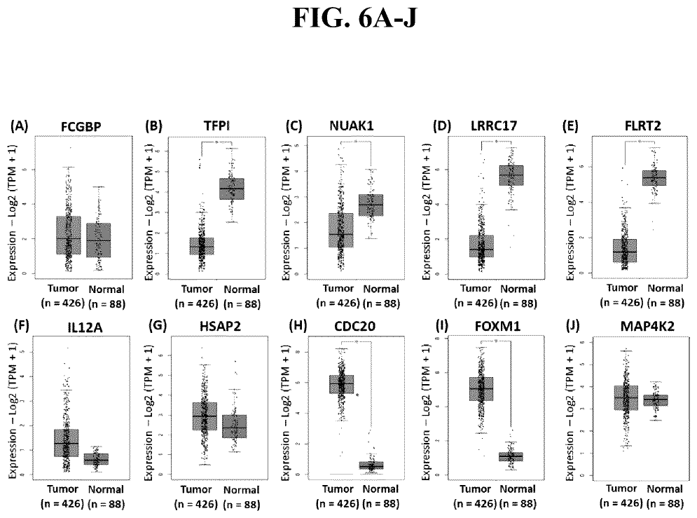

The same analysis was performed to validate the gene signatures identified for the platinum-only data. The mRNA expression of the gene signatures in normal and OC tissues was analyzed using GEPIA2 software to validate the expression of the identified gene signatures. These results indicated significantly higher mRNA levels of CDC20, and FOXM1 in OVCA compared to normal tissues (FIG. 6C-E,G,I). In contrast, there were significantly lower mRNA levels of TFPI, NUAK1, LRRC17, and FLRT2 in OVCA compared to normal tissues (FIG. 6F,H). Furthermore, FCGBP, MAP4K2, IL 12A, and HSPA2 showed similar expression levels in ovarian cancer compared to normal tissues (FIG. 6A, F, J).

### 2.5. Machine Learning Classification Performance

The performance evaluation of the models was conducted on both the training and validation sets using metrics such as accuracy, sensitivity, specificity, and AUC. Random forest was the best-performing machine learning algorithm, with the SVM algorithm following closely behind (Table 2).

TABLE 2
Classification method performance on training and validation sets of SOC patients
who were treated either with platinum-paclitaxel or platinum-only.

| Model | | | Accuracy (95% CI) ¹ | Sensitivity | Specificity | AUC ² |
|---|---|---|---|---|---|---|
| Platinum- | Training | Random Forest | 1 (0.92, 1) | 0.98 | 0.95 | 1 |
| Paclitaxel | (n = 64) | Support Vector Machine | 0.94 (0.82, 0.98) | 0.91 | 0.96 | 0.94 |
| | Validation | Random Forest | 0.93 (0.85, 0.95) | 0.90 | 0.94 | 0.93 |
| | (n = 16) | Support Vector Machine | 0.93 (0.85, 0.95) | 0.89 | 0.92 | 0.93 |
| Platinum-Only | Training | Random Forest | 0.99 (0.95, 1) | 1 | 0.99 | 0.99 |
| | (n = 74) | Support Vector Machine | 0.97 (0.90, 0.96) | 0.97 | 0.96 | 0.97 |
| | Validation | Random Forest | 0.95 (0.89, 0.97) | 0.95 | 0.94 | 0.94 |
| | (n = 19) | Support Vector Machine | 0.93 (0.84, 0.96) | 0.95 | 0.90 | 0.93 |

¹The values under the brackets correspond to 95% confidence interval (95% CI).
²AUC represents the area under curve.

For the platinum-paclitaxel training set, the random forest algorithm achieved an accuracy of 1 with a 95% confidence interval (CI) ranging between 0.92 and 1. The sensitivity and specificity were equal to 0.98 and 0.95, respectively. Support vector machine, on the other hand, achieved an accuracy of 0.94 with a 95% CI ranging between 0.82 and 0.98. The specificity and sensitivity were equal to 0.91 and 0.96, respectively. About the platinum-only training set, random forest algorithm achieved an accuracy of 0.99 with a 95% CI ranging between 0.95 and 1. The sensitivity and specificity were equal to 1 and 0.99, respectively. Support vector machine, on the other hand, achieved an accuracy of 0.97, with a 95% CI ranging between 0.90 and 0.96. The specificity and sensitivity were equal to 0.97 and 0.96, respectively (Table 2).

For the platinum-paclitaxel validation set, random forest algorithm had an accuracy of 0.91 with a 95% CI ranging between 0.77 and 0.96. In addition, sensitivity, specificity, and area under curve (AUC) were equal to 0.86, 0.92, and 0.91, respectively. The support vector machine algorithm obtained an accuracy of 0.91 which was equal to the value obtained from the random forest algorithm. In terms of sensitivity, specificity, and AUC, they were equal to 0.82, 0.92, and 0.90, respectively. On the other hand, for the platinum-only validation set, the random forest algorithm had an accuracy of 0.95 with a 95% CI ranging between 0.89 and 0.97. In addition, sensitivity, specificity, and area under curve (AUC) were equal to 0.95, 0.94, and 0.94, respectively. The support vector machine algorithm obtained an accuracy of 0.93 which was equal to the value obtained from the random forest algorithm. In terms of sensitivity, specificity, and AUC, they were equal to 0.95, 0.90, and 0.93, respectively (Table 2).

### 2.6. Biological Significance of the Identified Gene Signatures

To acquire an enhanced comprehension of the biological relevance of the gene signatures that were differentially expressed in SOC, the IPA software version (24.0) was used to subject the genes to analysis identifying the molecular processes that were significantly implicated. The ingenuity pathways knowledge base has provided pertinent insights on canonical pathways, diseases, and disorders, as well as molecular and cellular functions.

In terms of the canonical pathways, the nine genes are involved in glycine cleavage complex (p-value=3.49×10⁻³), germ cell-Sertoli cell junction signaling (p-value=4.28×10⁻³), tumor microenvironment pathway (p-value=4.73×10⁻³), and tumoricidal function of hepatic natural killer cells (p-value=1.39×10⁻²). In regard to the 10 genes associated with platinum-paclitaxel, they are involved in the following canonical pathways: the NOD1/2 signaling pathway (p-value=2.65×10⁻³), hepatic cholestasis (p-value=2.71×10⁻³), natural killer cell signaling (p-value=2.91×10⁻³), protein ubiquitination pathway (p-value=5.45×10⁻³), and extrinsic prothrombin activation pathway (p-value=6.64×10⁻³).

In terms of molecular and cellular functions, IPA demonstrated that the nine aforementioned genes are involved in cell-to-cell signaling and interaction (p-value range 1.09×10⁻²-1.63×10⁻⁷), cellular assembly and organization (p-value range 1.10×10⁻²-3.13×10⁻⁵), cellular function and maintenance (p-value range 8.13×10⁻³-3.13×10⁻⁵), cellular development (p-value range 9.8×10⁻²-4.22×10⁻⁵), and cellular growth and proliferation (p-value range 9.8×10⁻³-4.22×10⁻⁵). In regard to the 10 genes associated with platinum-paclitaxel, they are involved in the cell cycle (p-value=4.2×10⁻²-1.51×10⁻⁷), cellular development (p-value=4.40×10⁻²-1.51×10⁻⁷), cell death and survival (p-value=4.44×10⁻²-6.73×10⁻⁵), and cellular movement (p-value=4.76×10⁻²-9.20×10⁻⁵).

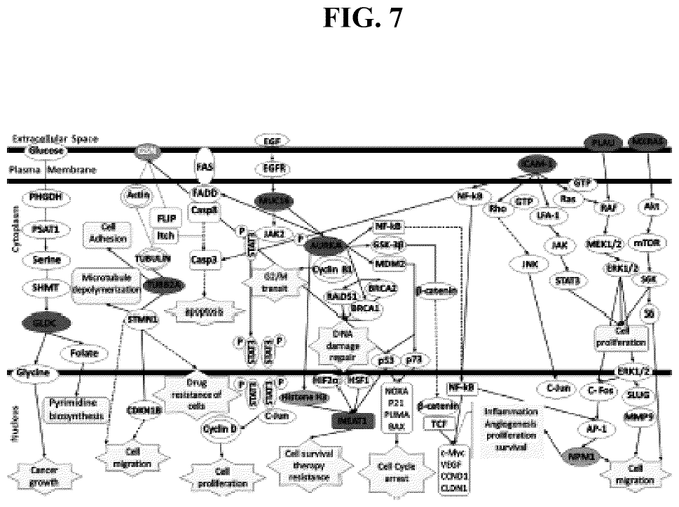

Furthermore, the generation of a schematic representation that illustrates the relationship between the gene signatures in different signaling pathways used the IPA library. FIG. 7 illustrates the pathways associated with the identified platinum-paclitaxel genes. GLDC was found to be involved in cancer growth through the PSAT1/Serine/SHMT/GLDC/Glycine complex. TUBB2A is associated with cell migration through STMN1/CDKN1B or cell adhesion via action. GSN was indirectly involved in the induction of apoptosis through the FLIP/Itch/Casp8 complex. MUC16 facilitates cell proliferation through the JAK2/STAT3/c-Jun/Cyclin D complex. It was also associated with G2/M cell cycle transition through the AURKA/Cyclin B1 complex. AURKA participates in many different cascades including NFκB, GSK3β, MDM2/p53, BRCA1/2, and RAD51. It is involved in cell cycle, inflammation, angiogenesis, cell proliferation and survival. NEAT1 is involved in cell cycle, cell survival and therapy resistance through Histone H3, p53, HIF2α, and HSF1. ICAM-1 participates in cell migration and proliferation via NFκB, NPM1, Pho/JNK, GTP/LFA-1/JAK/STAT3, and Ras/RAF/MEK/ERK activates the Raf/MEK/ERK pathway which is responsible for cancer cell proliferation and migration. PLAU has been observed to be associated with the Akt/mTOR/S6k pathway, which also activates cancer cell proliferation and migration.

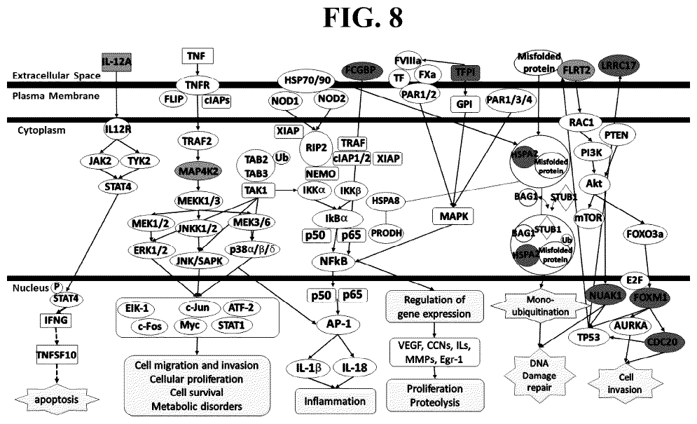

FIG. 8 illustrates the pathways associated with the identified platinum-paclitaxel genes. IL12a mediates signaling via either Jak2 or Tyk2, along with p-STAT4, IFNG, and TNFSF10, thereby contributing to the induction of apoptosis. MAP4K2 is activated through various transduction signals, including those initiated by TNF and TRAF2. MAP4K2 mediates signaling via MEKK1/3 activating the MEK/ERK, JNK/SAPK, and p38 α/β/δ pathways. These pathways activate multiple signals including EIK-1, c-Jun, ATF-2, c-Fos, Myc, and STAT1 which involve cell migration and invasion, cellular proliferation, cell survival, and metabolic disorders. TFPI was found to be associated with FVIIIa, FXa, TF, and PAR 1/2. TFPI mediates signaling via GPI and MAPK which, in turn, mediates NFκB signaling. HSPA2 is directly connected to HSP70/90 which, in turn, have an association with NOD1/2. NOD1/2 serves as a mediator for various signaling cascades that promote NFκB signaling. MEK/ERK, JNK/SAPK, p38 a/B/8, and NFκB pathways activate the AP-1 which subsequently promotes inflammation. NFκB pathway also stimulates proliferation and proteolysis by modulating the expression of key factors, including VEGF, CCNs, ILs, MMPs, and Egr-1. HSPA2 participates in DNA damage repair by modulating the expression of BAGI, STUB1, and the ubiquitination of misfolded protein. FLRT2 acts as a mediator by modulating the expression of RAC1/PTEN/PI3K/Akt/mTOR. Akt is involved in the activation of FOXO3a which, in turn, mediates the signaling of FOXM1 and CDC20. Both FOXM1 and CDC20 play a role in cell invasion. Akt also participates in the activation of NUAK1. Finally, both LRRC17 and NUAK1 affect TP53 expression.

### 2.7. Survival Analysis Using Kaplan-Meier

Survival analysis can assess the impact of gene signatures on the survival of SOC patients. Plotting the Kaplan-Meier survival curves for the gene signatures using the web-based curator considering lower and higher expression of genes and using the default parameters of a multiple hypothesis testing statistical method (p=0.05). The analysis followed KM plotter guidelines and opted for a Bonferroni correction threshold under 10% FDR to calculate significant analysis. Application of the Affymetrix ID of each gene further explored the prognostic potential of the gene signatures by assessing their correlation with histology type, grade, and chemotherapy type. A total of 1657 OC samples collected from GEO and TCGA databases were found in the KM Plotter database. The correlation between gene signatures expression and the clinical parameters was determined using univariate and multivariable Cox regression. The analysis was restricted to SOC samples who underwent debulking surgery and received either platinum-taxol or platinum-only chemotherapy. Further filtering of the data separated LGSOC (grade 1 and 2) from HGSOC (grade 3).

Regarding platinum-paclitaxel-related genes, ICAM-1 overexpression was associated with better OS in LGSOC, but no significant difference in LGSOC. TUBB2A, GLDC, and AURKA were associated with worse OS in all grades of SOC. While overexpression of PLAU and MXRA5 was only associated with worse OS in LGSOC, there was no significant difference in expression in HGSOC. In addition, there was no significant association between the expressions of NEAT1, GSN, and MUC16 and OS in all grades of SOC (Table 3).

TABLE 3
Subgroup analysis of gene signatures expression and patient prognosis according
to serous ovarian cancer grade treated with platinum-paclitaxel.

| | | Hazard | Median Survival (Months) | |
|---|---|---|---|---|
| Grade | Genes | Ratio | Low/High Expression | p-Value |
| Overall | ICAM1 | 0.55 (0.31-0.97) | 48/66.57 | p = 0.035 |
| Survival | TUBB2A | 1.96 (1.17-3.29) | 61.53/52 | p < 0.01 |
| (Grades | GLDC | 1.91 (1.21-3.04) | 66.57/48 | p < 0.01 |
| 1 and 2) | PLAU | 1.89 (1.13-3.18) | 57/48 | p = 0.014 |
| | AURKA | 1.74 (1.1-2.76) | 61.53/44.3 | p = 0.016 |
| | NEAT1 | 1.62 (0.93-2.83) | 83/52.63 | p = 0.086 |
| | MXRA5 | 1.71 (1.08-2.72) | 61.53/52.63 | p = 0.022 |
| | GSN | 0.82 (0.52-1.3) | 52.63/57 | p = 0.4 |
| | MUC16 | 1.38 (0.87-2.19) | 58/54.67 | p = 0.17 |
| Overall | ICAM1 | 1.12 (0.86-1.45) | 42.13/41.83 | p = 0.39 |
| Survival | TUBB2A | 1.42(1.12-1.8) | 45.77/38.47 | p < 0.01 |
| (Grade 3) | GLDC | 1.34 (1.04-1.74) | 45.23/41.6 | p = 0.025 |
| | PLAU | 0.78 (0.6-1.02) | 36.77/45.53 | p = 0.065 |
| | AURKA | 1.41 (1.1-1.81) | 48.37/38.73 | p < 0.01 |
| | NEAT1 | 1.24 (0.98-1.58) | 45.63/38.47 | p = 0.073 |
| | MXRA5 | 1.19 (0.92-1.54) | 42.6/41.83 | p = 0.19 |
| | GSN | 0.82 (0.63-1.08) | 41/45.23 | p = 0.17 |
| | MUC16 | 0.89 (0.7-1.15) | 38.4/44.8 | p = 0.38 |

With genes related to platinum-only treatment, the overexpression of HSPA2, NUAK1, LRRC17, and FLRT2 was associated with worse OS in all grades of SOC. FCGBP was associated with worse OS in HGSOC, but no significant difference in expression in LGSOC. On the other hand, there was no significant association between the expressions of TFPI, FOXM1, MAP4K2, CDC20, and IL12A and OS in all grades of SOC (Table 4).

TABLE 4
Subgroup analysis of gene signatures expression and patient prognosis
according to serous ovarian cancer treated with platinum-only.

| | | Hazard | Median Survival (Months) | |
|---|---|---|---|---|
| Grade | Genes | Ratio | Low/High Expression | p-Value |
| Overall | FCGBP | 1.23 (0.86-1.76) | 56.27/52.63 | p = 0.25 |
| Survival | HSPA2 | 1.54 (0.99-2.4) | 61.53/52.63 | p = 0.05 |
| (Grade | TFPI | 1.28 (0.89-1.83) | 57.1/48.27 | p = 0.18 |
| 1 + 2) | NUAK1 | 1.92 (1.33-2.76) | 60.5/37.13 | p < 0.01 |
| | LRRC17 | 1.84 (1.28-2.64) | 62.47/45.4 | p < 0.01 |
| | FLRT2 | 1.66 (1.15-2.39) | 59.33/48 | p < 0.01 |
| | FOXM1 | 0.77(0.54-1.1) | 48.27/57.33 | p = 0.14 |
| | MAP4K2 | 1.46 (0.96-2.21) | 65.17/52 | p = 0.073 |
| | CDC20 | 1.23 (0.86-1.75) | 57/48.37 | p = 0.26 |
| | IL12A | 1.27(0.89-1.83) | 57.1/50 | p = 0.18 |
| Overall | FCGBP | 1.47(1.2-1.79) | 46/35.77 | p < 0.01 |
| Survival | HSPA2 | 1.3 (1.08-1.58) | 46/38.77 | p < 0.01 |
| (Grade 3) | TFPI | 1.16 (0.95-1.41) | 45.77/41.57 | p = 0.14 |
| | NUAK1 | 1.39 (1.15-1.69) | 45.77/40.07 | p < 0.01 |
| | LRRC17 | 1.3 (1.07-1.57) | 45.77/38.43 | p < 0.01 |
| | FLRT2 | 1.26 (1.04-1.53) | 45.77/40.1 | p = 0.02 |
| | FOXM1 | 0.81 (0.65-1.01) | 42/48 | p = 0.058 |
| | MAP4K2 | 1.16 (0.95-1.4) | 45.53/41.57 | p = 0.14 |
| | CDC20 | 0.85 (0.69-1.05) | 38.4/45.47 | p = 0.13 |
| | IL12A | 0.86 (0.7-1.05) | 38.57/45.63 | p = 0.14 |

### 2.8. Machine Learning Model Application to Predict Effectiveness of Alternate Chemotherapy Regimen

The genes successfully classifying responders and non-responders for platinum-paclitaxel were different from the genes successfully classifying responders and non-responders for platinum-only suggesting different underlying mechanisms. It is possible that patients who did not respond to the combination of platinum-paclitaxel might respond to platinum-only treatment, and vice versa. Interestingly, the application of the random forest model for the platinum-paclitaxel dataset to cases of SOC treated with the platinum regimen, suggest that 34 of 93 (36.55%) SOC patients who did not respond to platinum-paclitaxel would respond to platinum-only treatment (Table 5). While this seems counter intuitive, there is evidence in previous studies, described in below section, that suggests that resistance can arise with the combination therapy that does not occur with the monotherapy case.

TABLE 5
Efficacy prediction for alternative treatment options.

| | Platinum- | Platinum- |
|---|---|---|
| | Paclitaxel | Paclitaxel |
| | Responder | Non-Responder |
| Responder with Platinum model | 25 (26.88%) | 34 (36.55%) |
| Non-responder with Platinum model | 16 (17.20%) | 18 (19.35%) |
| | Platinum | Platinum |
| | Responder | Non-Responder |
| Responder with | 0 (0.0%) | 0 (0.0%) |
| Platinum-Paclitaxel model | | |
| Non-responder with | 31 (38.75%) | 49 (61.25%) |
| Platinum-Paclitaxel model | | |

The presently disclosed study applied machine learning models and gene expression profiles to identify precise multi-gene panels that can predict the response to platinum-based chemotherapy, with or without the addition of paclitaxel, in SOC patients. The study demonstrates promising outcomes as a clinical indicator, showing a high level of accuracy. The random forest and support vector machine classifiers accurately classified responders' and non-responders' tumor samples in the GEO SOC validation sets with 91% accuracy for platinum-paclitaxel and 95% and 93% accuracy for platinum-only, respectively. The findings demonstrated that features identified by machine learning can distinguish resistant from sensitive tumors to the chemotherapy regimen. The results are similar to the results of two previous studies [16,48]. Their models also achieved accuracy above 90% in both the training and validation sets.

The mechanism of action of the platinum agents (i.e., cisplatin or carboplatin) is conditioned by the covalent binding of these molecules to DNA making DNA crosslinks and, eventually, inhibition of cell cycle and cell proliferation [49,50]. Some factors contributing to platinum resistance include overexpression of multidrug resistance proteins, advancement of DNA repair mechanisms, degradation, and deactivation of intracellular thiols [51]. The activation of cellular protective responses inhibits cell cycle progression to facilitate the repair of cisplatin-induced DNA damage [52]. Recognition of platinum-induced DNA damage occurs by diverse cellular mechanisms, including the MRE11-RAD50-NBSI complex, hMSH2 of the mismatch repair (MMR) complex, the nonhistone chromosomal high-mobility groups 1 and 2 proteins (HMG 1/2), and the transcription factor “TATA-binding protein” (TBP) [53]. These specific proteins recognize the damage and transmit signals to proteins such as p53, p73, and MAPK, leading to apoptosis and cell death [53]. MAPK signals (i.e., extracellular signal-related kinases (ERKs), c-Jun N-terminal kinases (JNKs), p38 kinases) play an important role in platinum-induced effects, with controversial data regarding their involvement in apoptosis. ERK activation induces p53 phosphorylation, resulting in cell cycle arrest, DNA damage repair, and activation of pro-apoptotic genes, ultimately leading to apoptosis. In addition, it has been shown that cisplatin induces p18 stabilization, which is a substrate of p38 kinases, increasing p53's ability to activate the transcription of proapoptotic genes such as PUMA and NOXA [53].

The mechanism of action of the taxane agents is the induction of cellular death by binding to tubulin and inhibiting the disassembly microtubules required for chromosome segregation and cell division. In addition, taxane treatment inhibits cell proliferation, induces apoptosis, and triggers diverse stress responses such as autophagy, senescence, and inflammation through complex mechanisms [53]. Similar to platinum-based chemotherapies, paclitaxel resistance develops in cancer cells via the efflux of paclitaxel out of the cells. Resistance to taxane agents can be attributed to PI3K/AKT hyperactivation including loss of function in PTEN and increases in anti-apoptotic Bcl-2 family members. The activation of these molecules can overpower the anti-proliferative signals leading to the upregulation of factors involved in cell proliferation and migration [54].

The genes identified in this study play crucial roles in the pathogenesis of serous-ovarian cancer and chemoresistance, offering significant biological insights and potential clinical applications. For instance, for platinum-paclitaxel, the study identified nine gene signatures associated with platinum-paclitaxel resistance, including ICAM1, TUBB2A, GLDC, PLAU, AURKA, NEAT1, MXRA5, MUC16, and GSN. These genes are involved in pathways that have been previously reported to be associated with chemoresistance in different cancers, including epithelial ovarian cancer. Particularly, it was found that overexpression of ICAM-1, MXRA5, AURKA, and NEAT1 are associated with the activation of NPM1, Histone H3, and TP53 in patients with serous ovarian cancer who received the platinum-paclitaxel chemotherapy regimen. A previous study showed that there is a link between an overexpression of nuclear NPM1 protein, chemoresistance, and poor outcomes for women diagnosed with HGSOC through the DNA repair function of APE1 and Ref-1 proteins [55]. APE1/Ref-1-NPM1 proteins are linked to cancer aggressiveness, which supports the idea that interfering with the APE1/Ref-1-NPM1 interaction might enable improved sensitization of cancer cells to chemotherapy [55]. Studies also showed that overexpression of ICAM-1 and AURKA increases the level of histone H3 [56,57]. The enhancer of zest homolog 2 (EZH2), a family member of the histone methyltransferases (HMT), can promote the cancer development through the catalyzation of the trimethylation of lysine at position 27 of histone H3, resulting in the suppression of downstream tumor suppressor genes [58]. Yang et al. (2020) reported that EZH2 was overexpressed in cisplatin-resistant OC cells compared to sensitive OC cells leading to blockage of cell death and proliferation of OC cells. ICAM-1 upregulation by the activation of multiple pathways including PKCα-p38-SP-1, JAK, PI3K, AKT, and NFκB has been observed previously [59]. The association of ICAM-1 overexpression was reported to be associated with reduced progression-free survival in SOC patients treated with platinum-paclitaxel compared to those treated with platinum-only, suggesting that patients with high ICAM1 expression might be resistant to Paclitaxel [59]. AURKA was found to be amplified in more than 15-25% of OC cell lines and primary tumors and to cause resistance to cisplatin by activating proteins such as p-eIF4E, c-MYC, HDM2, BRCA1/2 [60,61]. In addition, clinical data showed that patients with BRCA1/2 mutations respond better to cisplatin, leading to the hypothesis that AURKA has a synergistic effect with BRCA1/2 in platinum resistance [60]. AURKA has been identified to regulate many signaling pathways, such as the PI3K/Akt, mTOR, β-catenin/Wnt, and NFκB pathways, and tumorigenesis requires interactions among multiple signaling pathways [62]. The elevated MXRA5 was reported to be associated with tumor angiogenesis [63,64]. Bioinformatics studies and protein chip analyses identified an association between overexpressed MXRA5 and PI3K-Akt-mTOR cascade in pancreatic cancer cells [64]. MXRA5 is also upregulated in breast cancer and was found to be important for the EMT progression and matrix remodeling [64,65]. Previous studies showed that silencing NEAT1 inhibits the invasion of OC cells in vitro and attenuates tumor growth in vivo [66-68]. The knockdown of NEAT1 was associated with the increase in cisplatin-taxol sensitivity in MDA-MB-231 OC cells [69]. This study also reported that elevated NEAT1 expression and paraspeckle formation form part of such malignancy-associated stress response pathways such as p53 [69].

Furthermore, other previous studies showed a linkage between TUBB2A, PLAU, GLDC, and MUC16 and chemoresistance. TUBB2A, essential component of microtubules, related to growth, infiltration, and drug resistance in several different malignancies [70]. GLDC was found to enhance glycolysis and is highly expressed in tumor-initiating-cells in non-small cell lung carcinoma [71]. Interestingly however, Shin et al. (2018) reported that GLDC is downregulated in paclitaxel-resistant OC cells and was suggested to be associated with OC chemoresistance [72]. Kwon et al. (2015) reported that mitochondrial glycine synthesis, closely coupled to serine via a single reversible step catalyzed by serine hydroxy methyltransferase (SHMT), was associated with rapid cancer cell growth [71]. Further research on GLDC and drug resistance in OC is required to validate the results. The upregulated PLAU was associated with platinum-paclitaxel drug resistance and worse OS in LGSOC, but no significant difference in expression was observed in OS for HGSOC in the analysis. A recent study reported that PLAU overexpression promotes progression in ESCC and tumors including breast, bladder, and lung cancer [73]. Another study reported that the downregulation of PLAU reduces the EMT-related genes expressed in the oral squamous cell carcinoma (OSCC) cell line leading to cessation of cell migration and invasion [74]. PLAU promotes ESCC proliferation and tumor growth by activating the MAPK pathway [73]. MUC16 stimulates cell adhesion, growth, and metastasis, and evading attacks from natural killer cells aiding cancer cell progression [75]. A previous study reported that silencing MUC16 increased the sensitivity of OVCAR-3 cells to cisplatin and doxorubicin but not to paclitaxel [76]. Another study found that the overexpression of MUC16 induces breast cancer cell proliferation via its interaction with the non-receptor tyrosine kinase JAK2, and this interaction mediates phosphorylation of transcription factor STAT3, which may transactivate c-Jun for Cyclin DI expression [77]. Furthermore, decreased MUC16 expression results in an accumulation of breast cancer cells at the G2/M phase of the cell cycle via Cyclin B1 and phosphorylation of AURKA, which in turn leads to apoptosis of breast cancer cells through JNK signaling [77]. GSN participates in multiple important cellular signaling for motility, apoptosis, proliferation, differentiation, epithelial mesenchymal transition, and carcinogenesis phenotypes [78]. GSN plays roles as both the effecter and inhibitor of apoptosis, which underlines its association in a wide variety of cancer types [78]. A recent study by Arentz et al. (2023) found that overexpression of GSN was significantly associated with HGSOC patients treated with chemotherapy. Interestingly, another recent study contradicts the findings and the findings of Arentz et al. (2023), demonstrating that the expression and secretion of GSN were higher in chemo-resistant OC cells than in chemo-sensitive OC cells [80]. The supporting study by Onuma et al. (2022) suggested that higher levels of GSN prevent cisplatin from dissociating GSN from the FLIP-ITCH complex, thus preventing caspase-3 and -8 activation and caspase-mediated GSN cleavage and thereby inhibiting apoptosis in chemo-resistant OC cells. However, further analysis is needed to understand the role of GSN in OC.

For the platinum-only drug, the present study identified ten gene signatures associated with platinum-paclitaxel resistance including FCGBP, HSPA2, TFPI, NUAK1, LRRC17, FOXM1, CDC20, FLRT2, MAP4K2, and IL 12A. The analysis showed that these genes are associated with NOD1/2, natural killer, ubiquitin-proteosome, and tissue-factor-activated complex pathways. NOD1/2 act as an oncogene in ovarian cancer by upregulating immune-related pathways such as the RIPK2/NFκB signaling pathway [82]. Upregulation of these immune-related pathways seems to modulate several stress response systems eventually disrupting both proliferation and cellular migration via PI3K/Akt/mTOR, MAPK, TNF, and p53 signaling pathways [83]. For instance, inhibition of Akt confers resistance to cisplatin through p53-(FLICE)-like inhibitory protein (FLIP) interaction and FLIP ubiquitination, which was attenuated by p53 silencing [84,85]. For instance, the present study showed that the overexpression of NUAK1, LRRC17, FOXM1, and CDC20 as well as the downregulation of FLRT2 are associated with DNA repair pathway as well as tumor invasion pathways. Two previous studies have shown that NUAK1 overexpression was associated with platinum and taxane resistance in SOC patients. In addition, these studies reported a direct interaction between NUAK1/LKB1 and p53 pathway, as well as the NFκB pathway, particularly in HGSOC cells [86,87]. A recent study found that LRRC17, an inhibitor of the receptor activator of the NFκB ligand (RANKL), is a potent prognostic factor in SOC, demonstrating a significant correlation between the overexpression of LRRC17 and poor OS in SOC patients. In addition, the study suggested that overexpressed LRRC17 can inhibit chemotherapy-induced apoptosis in SOC [88]. The upregulated CDC20 was linked to platinum-only drug resistance in the analysis. CDC20 is one of the regulators of spindle checkpoint [89]. A previous study reported that CDC20 was remarkably upregulated by the knockdown of p53 [89]. The overexpression of CDC20 was significantly associated with SOC compared to the other types of OC. After silencing CDC20,EOC cell proliferation and migration decreased, and apoptosis increased [90]. FOXM1, on the other hand, has emerged as a multifunctional oncoprotein and a robust biomarker of poor prognosis in many human malignancies [91]. The FOXM1 transcriptional pathway was aberrantly activated in over 85% of cases and was rendered the second most frequent molecular alteration in HGSOC, second only to TP53 mutations [91]. Downregulation or inactivation of the p53 and Rb pathways results in the activation of the E2F1 transcription factor, which directly upregulates FOXM1 gene expression by binding to its promoter. These findings establish that p53 and Rb pathway dysregulation is a key contributor to FOXM1 overexpression in OC [91]. Finally, possible association of FLRT2 down-regulation with the process of ovarian and uterine cancers due to downregulated expression has been suggested [92]. Its biological function was verified only in prostate cancer and breast cancer [92], but its role in the tumorigenesis of OC remained unclear. Further research is needed to determine the role of FLRT2 in SOC.

Natural killer (NK) cells, which are lymphocytes of the innate immune system involved in the early defenses against foreign cells, express an array of activating cell surface receptors that can trigger cytolytic programs, as well as cytokine or chemokine secretion [93]. A recent study showed that carboplatin, one of the platinum agents, increased HLA-E, nectin-4, HLA-ABC, and CD111 expression in HGSOC cell lines, which was associated with an inhibitory NK receptor ligand phenotype [94]. Of these, nectin-4 was reported to have a role in HGSOC metastasis and chemotherapeutic resistance [94]. For instance, IL 12A, which was identified in the present study, was reported to play a critical role in the regulation of early inflammatory responses and promotion of the Thy 1-type repertoire [95]. IL-12A stimulates T-cells and NK cells to secrete IFN-y and increases the proliferation and cytolytic activity of these cells. IL-12A was reported to be an effective anti-cancer agent against various experimental malignancies [95].

The ubiquitin-proteasome pathway plays an important role in the regulation of cellular proteins involving cell cycle control, transcription, apoptosis, cell adhesion, angiogenesis, and tumor growth [96]. Various ways that the ubiquitin pathway is involved in OC, such as modulating the ovarian-cancer-related gene BRCA1 and tumor suppressor p53, and interfering with the ERK pathway, the cyclin-dependent cell cycle regulation process, and ERBB2 gene expression [96]. HSPA2, one of stress-non-inducible and least characterized members of the HSPA family (HSP70), is ubiquitous in various types of cancer cells [97]. HSP70 overexpression has been linked to ovarian cancer aggressiveness [98]. HSP70 has been shown to support tumor growth and invasion in EOC via modulating several cellular events including cell cycle, apoptosis, and epithelial mesenchymal transition pathways [98]. FCGBP has been found to be downregulated in many cancers including ovarian cancer [99]. It plays an important role in anti-inflammation and cell protection in epithelium cells as well as cell adhesion [99]. Cell adhesion occurring in the vasculature of specific organs is an essential step in cancer metastasis [99].

The tissue-factor-activated fVII (fVIIa) complex is an essential initiator of the extrinsic blood coagulation process [100]. Interactions between cancer cells and immune cells via coagulation factors and adhesion molecules can promote progression of cancer, including EOC [100]. TF, fVII, intercellular adhesion molecule-1 (ICAM-1), and multiple pro-inflammatory cytokines can be induced in response to hypoxia in EOC cancer cells at the gene expression level, leading to the autonomous production of the TF-fVII complex [100,101]. TFPI, a novel serodiagnostic marker for EOC, inhibits blood coagulation induced by tissue factor [102]. The diminished expression of TFPI could result in activated factor Xa and increase factor Xa-PAR2 signaling [103]. Various studies have suggested that therapeutic strategies that target an increase in the expression of TFPI could inhibit tumor angiogenesis, growth, and metastasis [103].

Finally, previous studies showed that 20 to 30% of OC patients fail to respond to the platinum-paclitaxel combination [104]. When patients progress on platinum-paclitaxel chemotherapy, it remains uncertain whether resistance has developed to one or both of the drugs, despite being labeled as platinum-paclitaxel-resistant [104]. The present findings were partially supported by previous studies [104,105]. Judson et al. evaluated the efficacy of combination drug therapy on cisplatin-resistant OC cells and found that cisplatin exerts mechanistic dominance over paclitaxel when human OC cells are simultaneously exposed to combination of cisplatin and paclitaxel [104]. This dominance adversely affects cisplatin-resistant cells by inhibiting paclitaxel-induced apoptosis [104]. Thus, suggesting that patients might derive significant benefits from a trial of paclitaxel alone as a second-line regimen in cases where initial treatment with cisplatin/paclitaxel has proven ineffective [104]. A recent study from Choi et al. supported the findings of Judson et al. [105]. They tested the effects of combined cisplatin and paclitaxel on cisplatin-resistant oral squamous cell carcinoma cells and found that cell growth was more inhibited by paclitaxel alone than combination therapy [105]. In addition, their study further suggests that the overexpression of FOXM1 protein by cisplatin makes it difficult to overcome drug resistance to cisplatin and causes resistance to paclitaxel, which can impact the effectiveness of combination therapy [105]. The data did not contain patients who received paclitaxel-only treatment to validate the results of these studies. Nonetheless, the disclosed findings concord with the results of Choi et al. As shown in FIG. 6, the data demonstrate that overexpression FOXM1 was associated with resistance to platinum in patients with SOC. Thus, it was speculated that this might be the underlying reason for the non-response observed in patients subjected to the platinum-paclitaxel model. Nevertheless, further clinical validation would be needed before this could influence clinical care.

From a clinical perspective, the identified gene signatures hold significant promise for improving serous ovarian cancer diagnosis and treatment. They can offer profound insights for enhancing diagnostic accuracy and tailoring personalized treatment strategies. These gene signatures could serve as biomarkers for the early detection of serous ovarian cancer, particularly in conditions where early intervention can significantly alter clinical outcomes. In addition, they have substantial potential in prognostic evaluations, providing clinicians with the ability to predict the progression of serous ovarian cancer and patient responses to platinum-based chemotherapy more precisely. The findings indicate that certain genes within these signatures are associated with resistance to platinum-based chemotherapy, a common treatment regimen for ovarian cancer. This resistance often leads to treatment failure and poor prognosis. By identifying patients who are genetically predisposed to this resistance, clinicians can avoid ineffective platinum-based therapies and instead opt for alternative treatment protocols that might be more effective. This preemptive approach not only spares patients from the side effects of ineffective treatment but also significantly reduces treatment costs and duration. Furthermore, understanding the mechanisms behind this resistance opens up ways for the development of new drugs aimed at modifying the expression or function of these resistant genes. For instance, novel inhibitors could be designed to target specific proteins encoded by the genes within the resistant signature, potentially restoring sensitivity to platinum-based treatments. This could revolutionize treatment protocols and improve survival rates for patients who would otherwise have limited options.

The LASSO and varSelR feature selection methods were selected because of their complementary strengths in handling high-dimensional data, which is a common characteristic of gene expression datasets. LASSO is particularly effective due to its ability to perform both variable selection and regularization simultaneously. This method helps in enhancing the prediction accuracy while reducing the complexity of the model by shrinking coefficients of less important variables to zero, thus effectively selecting a smaller subset of more relevant features. The ability of LASSO to impose a constraint on the model parameters makes it particularly suitable for models that suffer from multicollinearity which is a frequent issue in genomic data [106]. On the other hand, varSelRF is a non-linear approach using the random forest algorithm. Unlike LASSO, which is based on linear model, varSelRF is capable of capturing complex interactions between features, which is often required in understanding biological systems. The random forest algorithm provides an intrinsic ranking of feature importance based on how much feature decreases the purity of the node, allowing for effective identification of relevant biomarkers that might be missed by linear methods [107]. The aim was to use the linear and non-linear strengths of these methods by employing both LASSO and varSelRF, respectively. This approach allowed one to capture a broad spectrum of informative features in the analysis, thereby enhancing the biological relevance and robustness of the identified gene signatures. The combination of these methods ensures a more comprehensive analysis that could be achieved through a single method, especially in datasets where the underlying biological relationship can be complex and non-linear. This strategy has provided a balanced and rigorous approach to feature selection, offering a substantial justification for the selection and application of these specific methods in the study.

Challenges were encountered due to imbalanced classes and missing data that had to be addressed. To ensure the rigor and transparency of the study, close adherence to the PRISMA diagram for systematic reviews and meta-analysis was conducted, applying strict inclusion and exclusion criteria throughout the data selection process. The inclusion criteria required that datasets exclusively contain information pertinent to the cancer type and histology, specifically serous ovarian cancer and must be tissue samples. In addition, it was crucial that the datasets included detailed information about the experimental platform used and the drug responses outcomes. Stages of cancer were also an integral factor in the analysis. Samples lacking any of this essential information were excluded from further analysis. Following this systematic approach, the study was structured into two subsections based on the treatment regimen: one focusing on datasets of patients who received a combination of platinum-paclitaxel drugs, and the other on those treated with platinum-only. From the beginning, these datasets demonstrated an inherent imbalance in class sizes between responders and non-responders within each treatment category. To address this imbalance, several strategies were employed to mitigate overfitting without resorting to data balancing methods such as oversampling, which was avoided due to the inherent risk of introducing artificial bias and overfitting. Oversampling the minority class can lead to models that perform well on repeated synthetic samples but fail to generalize to new, read-world data. Instead, focus was on alternative approaches. First, algorithms less sensitive to class imbalance were used, such as tree-based methods including random forest, which inherently manage class disparity by focusing on data structure rather than frequency. Secondly, robust evaluation metrics were used such as specificity, sensitivity, and area under curve that provide a clearer indication of model performance across unbalanced classes. Stratified cross-validation was also implemented to ensure representative class distribution in each fold, enhancing model evaluation and stability. In addition, regularization techniques such as LASSO were applied to limit model complexity and prevent overlearning from the majority class. Finally, ensemble methods such as random forest were used to sequentially correct errors from previously built models, placing greater emphasis on previously misclassified instances, often from the minority class. These strategies collectively helped in reducing the risk of overfitting while improving model robustness and accuracy across the unbalanced datasets.

A strength of this study is that previous prediction studies included patients with varying clinical characteristics and histological types of OC, which made generalizability difficult. In this study, to ensure the validity of the research, only patients diagnosed with SOC were included. One limitation of the study is the sample size of the overall data. To mitigate the risk of overfitting that can be caused due to the small sample size, several strategies were implemented. The K-fold cross-validation across the training and validation set was applied to ensure that the performance of the models is consistently evaluated against multiple data splits, enhancing the generalizability of the findings. Feature selection methods were also applied to ensure that only relevant predictors were included, minimizing the chance of the model capturing irrelevant variability.

Despite the potential constraints posed by the sample size, it is crucial to recognize the importance of accurately defining outcomes and ensuring homogeneity within the population when constructing prediction models. These factors must take precedence in order to produce reliable and valid results.

In conclusion, the current study found gene signatures capable of making high-accuracy prediction of the response to platinum-based chemotherapy in patients with serous ovarian cancer. This machine learning approach predicts a useful approach for improving drug treatment outcomes for cancer patients. This approach has significant potential for integration into clinical practice after additional clinical validation.

Analysis of the gene signatures gives the following insights into the important mechanisms for platinum-paclitaxel resistance in both low-and high-grade serous ovarian cancer. The non-responders to the drug seem to have genes including ICAM1, TUBB2A, GLDC, PLAU, AURKA, NEAT1, MXRA5, GSN, and MUC16 that promote cancer growth and cell proliferation through dysregulation of JAK2, STAT3, MAPK, AKT, and mTOR as well as DNA damage via BRCA1/2 and TP53. These genes are associated with pathways such as glycine cleavage complex and tumor microenvironment known to be linked to chemoresistance in many cancers including OC.

The analysis of gene signatures associated with platinum-only creates insights into the important mechanisms for platinum resistance in both low-and high-grade SOC. The change in expression in the following genes, FCGBP, TFPI, NUAK1, LRRC17, FLRT2, IL 12A, HSPA2, CDC20, MAP4K2, and FOXM1, results in cell proliferation and invasion via aberration of JAK2, STAT4, and MAPK as well as apoptosis inhibition via TP53, AURKA, and NFκB. These genes have been previously reported to be associated with pathways involved in chemoresistance in OC including NOD1/2, natural killers (NK), ubiquitin-proteasome, and the tissue-factor-activated fVII complex.

Finally, the analysis as well as previous research demonstrated that overexpression of FOXM1 was associated with resistance to platinum in patients with SOC. Thus, this might be the underlying reason for the non-response observed in patients subjected to the platinum-paclitaxel model.

TABLE 6
Differentially expressed genes identified between responders and non-responders
of platinum-paclitaxel in patients with serous ovarian cancer.

| Gene.symbol | logFC | AveExpr | t | P.Value | log2FoldChange | P.Adjusted Value |
|---|---|---|---|---|---|---|
| ICAM1 | 1.258712 | 4.712829 | 3.825611 | 0.000355 | 2.39282 | 0.021741 |
| NEAT1 | 1.750267 | 7.9721 | 3.660446 | 0.000594 | 3.364209 | 0.021741 |
| MUC4 | 1.870482 | 8.740564 | 3.585137 | 0.000749 | 3.656548 | 0.021741 |
| CAPZB | 1.102599 | 4.822414 | 3.564364 | 0.000798 | 2.147411 | 0.021741 |
| THSD4 | 1.945427 | 6.571429 | 3.486054 | 0.001012 | 3.851518 | 0.022059 |
| LOC105370109 | 1.325439 | 4.488578 | 3.299423 | 0.001764 | 2.506091 | 0.029612 |
| MXRA5 | 1.303457 | 4.711662 | 3.233476 | 0.002138 | 2.468197 | 0.029612 |
| CALR | 1.06549 | 6.106294 | 3.169809 | 0.002569 | 2.092881 | 0.029612 |
| RDH13 | 1.409014 | 4.5503 | 3.126192 | 0.002911 | 2.655556 | 0.029612 |
| MUC16 | 1.259785 | 4.347569 | 3.120342 | 0.00296 | 2.3946 | 0.029612 |
| PLAU | 1.059728 | 3.07657 | 3.116989 | 0.002988 | 2.084539 | 0.029612 |
| TUBB2A | 1.510156 | 6.142812 | 3.073643 | 0.003379 | 2.848408 | 0.030695 |
| DCHS1 | 1.119577 | 5.361855 | 3.015963 | 0.003974 | 2.172832 | 0.032199 |
| ITGB5 | 1.724056 | 7.110435 | 2.966262 | 0.004564 | 3.303639 | 0.032199 |
| NRBP2 | 1.095633 | 5.895585 | 2.957295 | 0.004678 | 2.137068 | 0.032199 |
| TBL1XR1 | 1.513537 | 7.706752 | 2.941258 | 0.00489 | 2.855091 | 0.032199 |
| GLDC | 1.085652 | 3.396137 | 2.888187 | 0.005656 | 2.122335 | 0.032199 |
| PAN3 | 1.179783 | 6.569144 | 2.885462 | 0.005699 | 2.265426 | 0.032199 |
| IFT80 | 1.226002 | 4.825622 | 2.884846 | 0.005708 | 2.339179 | 0.032199 |
| MRI1 | 1.081992 | 5.414245 | 2.85229 | 0.006236 | 2.116957 | 0.032199 |
| PARD6B | 1.04961 | 5.473988 | 2.835552 | 0.006525 | 2.06997 | 0.032199 |
| SRRM2 | 1.245625 | 4.85799 | 2.835388 | 0.006528 | 2.371212 | 0.032199 |
| SNX19 | 1.104696 | 4.528653 | 2.806314 | 0.00706 | 2.150536 | 0.032199 |
| TIPRL | 1.575885 | 6.295907 | 2.804731 | 0.00709 | 2.981183 | 0.032199 |
| TIMP3 | 1.265991 | 5.549762 | 2.780104 | 0.007573 | 2.404924 | 0.033018 |
| CDR1 | 1.656838 | 6.518186 | 2.747068 | 0.008269 | 3.153246 | 0.033257 |
| SFT2D2 | 1.244536 | 6.663114 | 2.729754 | 0.008657 | 2.369423 | 0.033257 |
| AEBP1 | 1.367908 | 8.949637 | 2.725802 | 0.008748 | 2.58096 | 0.033257 |
| SRSF11 | 1.015878 | 6.647739 | 2.721468 | 0.008848 | 2.022133 | 0.033257 |
| CD81 | 1.035816 | 7.181112 | 2.679819 | 0.009871 | 2.050273 | 0.033756 |
| RBPMS | 1.305834 | 6.510124 | 2.679614 | 0.009876 | 2.472266 | 0.033756 |
| HSPA5 | 1.101874 | 7.735995 | 2.667748 | 0.010187 | 2.146332 | 0.033756 |
| PGLS | 1.094452 | 10.30997 | 2.666511 | 0.01022 | 2.135319 | 0.033756 |
| PPIC | 1.231994 | 7.191868 | 2.623606 | 0.011423 | 2.348915 | 0.034372 |
| ITM2B | 1.072594 | 6.989541 | 2.617335 | 0.011609 | 2.103211 | 0.034372 |
| NNMT | 1.736113 | 8.017737 | 2.617328 | 0.01161 | 3.331364 | 0.034372 |
| GSN | −1.11553 | 5.396278 | −2.61539 | 0.011668 | −2.16675 | 0.034372 |
| SNX21 | 1.12754 | 4.674479 | 2.599682 | 0.012149 | 2.184858 | 0.034848 |
| ESD | 1.08948 | 5.665136 | 2.568969 | 0.013143 | 2.127973 | 0.036732 |
| LAMP1 | 1.407333 | 10.41783 | 2.542265 | 0.014066 | 2.652464 | 0.037877 |
| VCAN | 1.092599 | 4.240653 | 2.530426 | 0.014494 | 2.132578 | 0.037877 |
| WNT6 | 1.992857 | 6.644343 | 2.527673 | 0.014595 | 3.980244 | 0.037877 |
| COL11A1 | 1.039985 | 3.259803 | 2.514979 | 0.01507 | 2.056206 | 0.0382 |
| SPARC | 1.113733 | 7.319859 | 2.494076 | 0.015882 | 2.164049 | 0.038537 |
| CDC42BPA | 1.068818 | 6.924712 | 2.493389 | 0.01591 | 2.097713 | 0.038537 |
| NRP2 | 1.044649 | 6.783994 | 2.441491 | 0.018105 | 2.062864 | 0.0429 |
| CHI3L1 | 1.495498 | 8.68154 | 2.421308 | 0.01903 | 2.819614 | 0.043454 |
| LUC7L3 | 1.412171 | 8.337008 | 2.417485 | 0.019209 | 2.661374 | 0.043454 |
| COL5A2 | 1.122494 | 6.9421 | 2.405 | 0.019808 | 2.17723 | 0.043454 |
| ABCC10 | 1.017467 | 4.527186 | 2.399509 | 0.020076 | 2.024362 | 0.043454 |
| LENG8 | 1.211602 | 10.24423 | 2.388984 | 0.020599 | 2.315947 | 0.043454 |
| AURKA | 1.109982 | 6.450443 | 2.386393 | 0.02073 | 2.158429 | 0.043454 |
| MRPL57 | 1.152862 | 6.191982 | 2.37146 | 0.021498 | 2.223546 | 0.044214 |
| SLC34A2 | 1.457094 | 5.979735 | 2.353086 | 0.022478 | 2.745548 | 0.044216 |
| SPON1 | 1.422028 | 7.917827 | 2.341612 | 0.023111 | 2.67962 | 0.044216 |
| CP | 1.345019 | 6.907464 | 2.339052 | 0.023254 | 2.540336 | 0.044216 |
| ARHGAP33 | 1.044922 | 5.432901 | 2.320916 | 0.024291 | 2.063255 | 0.044216 |
| SIDT2 | 1.30597 | 8.080496 | 2.318244 | 0.024447 | 2.472499 | 0.044216 |
| ANXA2 | 1.038303 | 7.476907 | 2.315426 | 0.024613 | 2.05381 | 0.044216 |
| XAF1 | 1.048172 | 6.313692 | 2.313144 | 0.024748 | 2.067909 | 0.044216 |
| ADAMTS10 | −1.07193 | 5.088951 | −2.30798 | 0.025056 | −2.10225 | 0.044216 |
| COL3A1 | 1.475464 | 6.934609 | 2.306405 | 0.025151 | 2.78073 | 0.044216 |
| KLK7 | 1.154481 | 6.604088 | 2.288665 | 0.026238 | 2.226042 | 0.044433 |
| ZNF503 | 1.003374 | 7.024202 | 2.287067 | 0.026338 | 2.004683 | 0.044433 |
| PTX3 | 1.069121 | 3.331447 | 2.284545 | 0.026497 | 2.098155 | 0.044433 |
| AHNAK2 | 1.010325 | 6.487022 | 2.26623 | 0.027673 | 2.014364 | 0.045252 |
| USP4 | 1.112193 | 6.038708 | 2.26406 | 0.027816 | 2.161739 | 0.045252 |
| FN1 | 1.457346 | 6.029537 | 2.249265 | 0.028804 | 2.746026 | 0.046171 |
| ACACB | 1.045382 | 6.943146 | 2.20679 | 0.031817 | 2.063912 | 0.050039 |
| HPS3 | 1.07159 | 5.923856 | 2.20202 | 0.032173 | 2.101748 | 0.050039 |
| COX5A | 1.103853 | 8.719836 | 2.196418 | 0.032594 | 2.149279 | 0.050039 |

TABLE 7
Differential expressed genes identified between responders and non-
responders of platinum-only in patients with serous ovarian cancer.

| Gene.symbol | logFC | AveExpr | t | P.Value | log2FoldChange | P.Adjusted Value |
|---|---|---|---|---|---|---|
| GNG11 | 1.335782 | 8.696821 | 3.878534 | 0.000377 | 2.524123 | 0.015882 |
| HSPA2 | 1.658692 | 9.024278 | 3.786319 | 0.000496 | 3.157302 | 0.015882 |
| ARHGAP6 | 1.603253 | 6.274051 | 3.72114 | 0.000601 | 3.038277 | 0.015882 |
| NCAPH | −1.43792 | 6.025163 | −3.6898 | 0.000659 | −2.7093 | 0.015882 |
| EPS8 | 1.167717 | 9.87811 | 3.573949 | 0.000925 | 2.246558 | 0.015882 |
| LMNB1 | −1.1388 | 8.587725 | −3.57369 | 0.000925 | −2.20197 | 0.015882 |
| TFPI | 1.700585 | 5.716612 | 3.562079 | 0.000957 | 3.250328 | 0.015882 |
| ECM2 | 1.951281 | 6.220575 | 3.526755 | 0.00106 | 3.867176 | 0.015882 |
| FOXM1 | 1.410511 | 7.744498 | −3.49561 | 0.001159 | 2.658314 | 0.015882 |
| PDPN | 1.99796 | 6.324915 | 3.493535 | 0.001166 | 3.994349 | 0.015882 |
| BICC1 | 2.126621 | 6.755107 | 3.482709 | 0.001203 | 4.366936 | 0.015882 |
| DCHS1 | 1.283424 | 7.140502 | 3.451484 | 0.001316 | 2.43416 | 0.015882 |
| MAP4K2 | −1.19892 | 5.409862 | −3.44977 | 0.001322 | −2.29568 | 0.015882 |
| FBXL7 | 1.311645 | 7.394482 | 3.432115 | 0.00139 | 2.482244 | 0.015882 |
| JAM3 | 1.264863 | 7.682606 | 3.426537 | 0.001413 | 2.403044 | 0.015882 |
| POLE2 | −1.10828 | 6.01334 | −3.4219 | 0.001431 | −2.15588 | 0.015882 |
| PLS3 | 1.070689 | 11.39187 | 3.416957 | 0.001452 | 2.100436 | 0.015882 |
| GEM | 1.853742 | 5.955311 | 3.25211 | 0.002309 | 3.614366 | 0.023858 |
| LHFPL6 | 1.263463 | 7.603347 | 3.20711 | 0.002616 | 2.400713 | 0.02525 |
| DACT1 | 1.526019 | 3.8142 | 3.174369 | 0.002863 | 2.879901 | 0.02525 |
| ZFP36 | 1.159976 | 9.974942 | 3.147939 | 0.003079 | 2.234536 | 0.02525 |
| NUAK1 | 1.146238 | 8.943608 | 3.119241 | 0.00333 | 2.21336 | 0.02525 |
| PSRC1 | −1.15782 | 8.015279 | −3.0935 | 0.003572 | 2.2312 | 0.02525 |
| MIS18BP1 | −1.07816 | 6.606418 | −3.08367 | 0.003669 | −2.11135 | 0.02525 |
| RAD54L | −1.15361 | 6.50937 | −3.07485 | 0.003758 | 2.22471 | 0.02525 |
| CLIP2 | 1.326334 | 6.685706 | 3.067972 | 0.003829 | 2.507646 | 0.02525 |
| LRRC17 | 1.44434 | 6.336302 | 3.066047 | 0.003849 | 2.721383 | 0.02525 |
| LMOD1 | 1.359963 | 5.034277 | 3.059526 | 0.003917 | 2.566787 | 0.02525 |
| NID2 | 1.721564 | 8.862003 | 3.048086 | 0.00404 | 3.297938 | 0.02525 |
| HJURP | −1.06285 | 7.933834 | −3.03712 | 0.004161 | −2.08905 | 0.02525 |
| PIMREG | −1.16897 | 6.688907 | −3.02232 | 0.004331 | −2.24851 | 0.02525 |
| CDC7 | −1.00892 | 8.028834 | −3.02116 | 0.004344 | −2.01241 | 0.02525 |
| LDB2 | 1.096171 | 7.0959 | 3.000146 | 0.004596 | 2.137865 | 0.025906 |
| FHL2 | 1.154055 | 10.12646 | 2.948715 | 0.005272 | 2.225385 | 0.02715 |
| KIT | 1.125145 | 4.351403 | 2.94574 | 0.005314 | 2.181235 | 0.02715 |
| PDZRN3 | 1.368351 | 9.336317 | 2.923579 | 0.005636 | 2.581752 | 0.02715 |
| AURKB | −1.13456 | 6.962698 | −2.92305 | 0.005643 | −2.19552 | 0.02715 |
| FLRT2 | −1.49155 | 7.839063 | 2.919708 | 0.005694 | −2.8119 | 0.02715 |
| TGFB1I1 | 1.176101 | 8.014173 | 2.907715 | 0.005877 | 2.259653 | 0.02715 |
| ABCA8 | 2.113317 | 5.243902 | 2.904782 | 0.005922 | 4.326848 | 0.02715 |
| PAQR4 | −1.15376 | 7.254754 | −2.88609 | 0.006221 | −2.22494 | 0.02715 |
| P3H2 | 1.641742 | 6.445609 | 2.88606 | 0.006222 | 3.120425 | 0.02715 |
| CDCA8 | −1.22406 | 7.082655 | −2.8799 | 0.006323 | −2.33603 | 0.02715 |
| CENPA | −1.07169 | 8.194276 | −2.87311 | 0.006437 | −2.10189 | 0.02715 |
| BAMBI | 1.66729 | 5.998542 | 2.86537 | 0.006569 | 3.176173 | 0.02715 |
| PLXDC1 | 1.106742 | 6.375125 | 2.851462 | 0.006812 | 2.153588 | 0.027544 |
| GPD2 | −1.10623 | 6.036807 | −2.83477 | 0.007115 | −2.15282 | 0.028158 |
| DUSP1 | 1.343239 | 11.50743 | 2.814423 | 0.007502 | 2.537203 | 0.028355 |
| OSR2 | 1.411669 | 7.166591 | 2.809057 | 0.007607 | 2.660447 | 0.028355 |
| RGS2 | 1.096386 | 8.905245 | 2.807224 | 0.007643 | 2.138183 | 0.028355 |
| SPINK5 | −1.68576 | 6.557589 | −2.80007 | 0.007786 | −3.2171 | 0.028355 |
| IGFBP4 | 1.52483 | 8.92931 | 2.787523 | 0.008043 | 2.877527 | 0.028355 |
| HTRA1 | 1.145164 | 10.46277 | 2.784957 | 0.008096 | 2.211713 | 0.028355 |
| OLFML1 | 1.233281 | 4.661019 | 2.776683 | 0.008271 | 2.35101 | 0.028355 |
| CORO2B | 1.502275 | 5.101271 | 2.771377 | 0.008384 | 2.83289 | 0.028355 |
| NR2F1 | 1.307398 | 8.724593 | 2.727676 | 0.009379 | 2.474947 | 0.03115 |
| CCN5 | 1.30084 | 2.892162 | 2.70548 | 0.009924 | 2.463724 | 0.032384 |
| CHST15 | 1.096118 | 9.57933 | 2.689503 | 0.010335 | 2.137787 | 0.033143 |
| SNAP91 | 1.018635 | 2.645548 | 2.681902 | 0.010536 | 2.026001 | 0.033194 |
| NDNF | 1.3546 | 4.075845 | 2.675501 | 0.010708 | 2.557261 | 0.033194 |
| EMILIN1 | 1.241812 | 6.880695 | 2.668303 | 0.010904 | 2.364954 | 0.033198 |
| ZFP69B | −1.14496 | 4.040907 | −2.66247 | 0.011066 | −2.2114 | 0.033198 |
| CDC20 | 1.029583 | 9.715055 | −2.65024 | 0.011412 | 2.041435 | 0.033692 |
| TXK | −1.29051 | 3.615809 | −2.63124 | 0.011969 | −2.44614 | 0.034698 |
| GFPT2 | 1.128325 | 4.967894 | 2.626064 | 0.012126 | 2.186047 | 0.034698 |
| FCGBP | 1.512422 | 7.911767 | 2.619804 | 0.012317 | 2.852887 | 0.034711 |
| CCN2 | 1.291488 | 10.1563 | 2.602626 | 0.012856 | 2.447804 | 0.035691 |
| NBL1 | 1.226406 | 8.97529 | 2.575831 | 0.013741 | 2.339834 | 0.037586 |
| GHR | 1.354641 | 4.232007 | 2.567713 | 0.01402 | 2.557334 | 0.037604 |
| IL12A | −1.22332 | 3.273814 | −2.55717 | 0.01439 | −2.33483 | 0.037604 |
| KLF2 | 1.067341 | 8.37353 | 2.554954 | 0.014469 | 2.095567 | 0.037604 |
| ANGPTL4 | 1.048256 | 5.368723 | 2.552499 | 0.014557 | 2.068029 | 0.037604 |
| NR4A1 | 1.322153 | 6.932428 | 2.512722 | 0.016049 | 2.500389 | 0.040893 |
| EGR2 | 1.178688 | 7.78475 | 2.506513 | 0.016295 | 2.263709 | 0.040957 |
| FAT1 | 1.000169 | 10.04318 | 2.497491 | 0.016657 | 2.000235 | 0.04131 |
| ACTA2 | 1.137667 | 11.18711 | 2.483609 | 0.017229 | 2.20025 | 0.041657 |
| PDGFRA | 1.567588 | 9.429396 | 2.483231 | 0.017245 | 2.964086 | 0.041657 |
| ASF1B | −1.06013 | 7.587243 | −2.4611 | 0.018195 | −2.08512 | 0.042503 |
| SYCP2 | −1.32573 | 5.055069 | −2.45997 | 0.018244 | −2.5066 | 0.042503 |
| FLG | 1.448872 | 2.972198 | 2.455855 | 0.018427 | 2.729945 | 0.042503 |
| H3C10 | −1.50607 | 5.234114 | −2.454 | 0.018509 | −2.84035 | 0.042503 |
| KIF18B | −1.01308 | 8.053595 | −2.38514 | 0.021827 | −2.01822 | 0.04951 |

### REFERENCES

1. Sung H; Ferlay J; Siegel R L; Laversanne M; Soerjomataram I; Jemal A; Bray F Global Cancer Statistics 2020: GLOBOCAN Estimates of Incidence and Mortality Worldwide for 36 Cancers in 185 Countries. CA Cancer J. Clin. 2021, 71, 209-249. [PubMed: 33538338]

2. Siegel R L; Miller K D; Fuchs H E; Jemal A Cancer statistics, 2022. CA Cancer J. Clin. 2022, 72, 7-33. [PubMed: 35020204]

3. Prat J New insights into ovarian cancer pathology. Ann. Oncol. 2012, 23, x111-x117. [PubMed: 22987944]

4. Garzon S; Laganà A S; Casarin J; Raffaelli R; Cromi A; Franchi M; Barra F; Alkatout I; Ferrero S; Ghezzi F Secondary and tertiary ovarian cancer recurrence: What is the best management? Gland. Surg. 2020, 9, 1118-1129. [PubMed: 32953627]

5. McCluggage W G Morphological subtypes of ovarian carcinoma: A review with emphasis on new developments and pathogenesis. Pathology 2011, 43, 420-432. [PubMed: 21716157]

6. Guadagno E; Pignatiello S; Borrelli G; Cervasio M; Della Corte L; Bifulco G; Insabato L Ovarian borderline tumors, a subtype of neoplasm with controversial behavior. Role of Ki67 as a prognostic factor. Pathol.-Res. Pract. 2019, 215, 152633. [PubMed: 31542184]

7. Reade C J; McVey R M; Tone A A; Finlayson S J; McAlpine J N; Fung-Kee-Fung M; Ferguson S E The fallopian tube as the origin of high grade serous ovarian cancer: Review of a paradigm shift. J. Obstet. Gynaecol. Can. 2014, 36, 133-140. [PubMed: 24518912]

8. Atallah G A; Kampan N C; Chew K T; Mohd Mokhtar N; Md Zin R R; Shafiee M. N. b.; Aziz N. H. b.A. Predicting Prognosis and Platinum Resistance in Ovarian Cancer: Role of Immunohistochemistry Biomarkers. Int. J. Mol. Sci. 2023, 24, 1973. [PubMed: 36768291]

9. Vang R; Shih I-M; Kurman R J Ovarian low-grade and high-grade serous carcinoma: Pathogenesis, clinicopathologic and molecular biologic features, and diagnostic problems. Adv. Anat. Pathol. 2009, 16, 267. [PubMed: 19700937]

10. Torre L A; Trabert B; DeSantis C E; Miller K D; Samimi G; Runowicz C D; Gaudet M M; Jemal A; Siegel R L Ovarian cancer statistics, 2018. CA Cancer J. Clin. 2018, 68, 284-296. [PubMed: 29809280]

11. Wang E W; Wei CH; Liu S; Lee S J-J; Shehayeb S; Glaser S; Li R; Saadat S; Shen J; Dellinger T Frontline Management of Epithelial Ovarian Cancer-Combining Clinical Expertise with Community Practice Collaboration and Cutting-Edge Research. J. Clin. Med. 2020, 9, 2830. [PubMed: 32882942]

12. Cannistra S A Cancer of the ovary. N. Engl. J. Med. 2004, 351, 2519-2529. [PubMed: 15590954]

13. Friedlander M; Matulonis U; Gourley C; du Bois A; Vergote I; Rustin G; Scott C; Meier W; Shapira-Frommer R; Safra T; et al. Long-term efficacy, tolerability and overall survival in patients with platinum-sensitive, recurrent high-grade serous ovarian cancer treated with maintenance olaparib capsules following response to chemotherapy. Br. J. Cancer 2018, 119, 1075-1085. [PubMed: 30353045]

14. Therasse P; Arbuck S G; Eisenhauer E A; Wanders J; Kaplan R S; Rubinstein L; Verweij J; Van Glabbeke M; van Oosterom A T; Christian M C; et al. New guidelines to evaluate the response to treatment in solid tumors. European Organization for Research and Treatment of Cancer, National Cancer Institute of the United States, National Cancer Institute of Canada. J. Natl. Cancer Inst.2000, 92, 205-216. [PubMed: 10655437]

15. Friedlander M; Butow P; Stockler M; Gainford C; Martyn J; Oza A; Donovan H S; Miller B; King M Symptom control in patients with recurrent ovarian cancer: Measuring the benefit of palliative chemotherapy in women with platinum refractory/resistant ovarian cancer. Int. J. Gynecol. Cancer 2009, 19 (Suppl. 2), S44-S48. [PubMed: 19955914]

16. Gonzalez Bosquet J; Devor E J; Newtson A M; Smith BJ; Bender D P; Goodheart M J; McDonald M E; Braun T A; Thiel K W; Leslie KK Creation and validation of models to predict response to primary treatment in serous ovarian cancer. Sci. Rep. 2021, 11, 5957. [PubMed: 33727600]

17. Walker J L; Brady M F; Wenzel L; Fleming G F; Huang HQ; DiSilvestro P A; Fujiwara K; Alberts D S; Zheng W; Tewari K S; et al. Randomized Trial of Intravenous Versus Intraperitoneal Chemotherapy Plus Bevacizumab in Advanced Ovarian Carcinoma: An NRG Oncology/Gynecologic Oncology Group Study. J. Clin. Oncol. 2019, 37, 1380-1390. [PubMed: 31002578]

18. Baekelandt M; Kristensen G B; Nesland J M; Tropé C G; Holm R Clinical significance of apoptosis-related factors p53, Mdm2, and Bcl-2 in advanced ovarian cancer. J. Clin. Oncol. 1999, 17, 2061. [PubMed: 10561259]

19. Baekelandt M; Holm R; Nesland J M; Trope C G; Kristensen G B Expression of apoptosis-related proteins is an independent determinant of patient prognosis in advanced ovarian cancer. J. Clin. Oncol. 2000, 18, 3775-3781. [PubMed: 11078490]

20. Abu Samaan T M; Samec M; Liskova A; Kubatka P; B usselberg D Paclitaxel's Mechanistic and Clinical Effects on Breast Cancer. Biomolecules 2019, 9, 789. [PubMed: 31783552]

21. Weaver B A How Taxol/paclitaxel kills cancer cells. Mol. Biol. Cell 2014, 25, 2677-2681. [PubMed: 25213191]

22. Nezi L; Musacchio A Sister chromatid tension and the spindle assembly checkpoint. Curr. Opin. Cell Biol. 2009, 21, 785-795. [PubMed: 19846287]

23. Lu T-P; Kuo K-T; Chen C-H; Chang M-C; Lin H-P; Hu Y-H; Chiang Y-C; Cheng W-F; Chen C-A Developing a Prognostic Gene Panel of Epithelial Ovarian Cancer Patients by a Machine Learning Model. Cancers 2019, 11, 270. [PubMed: 30823599]

24. Yu K-H; Levine D A; Zhang H; Chan DW; Zhang Z; Snyder M Predicting Ovarian Cancer Patients' Clinical Response to Platinum-Based Chemotherapy by Their Tumor Proteomic Signatures. J. Proteome Res. 2016, 15, 2455-2465. [PubMed: 27312948]

25. Amniouel S; Jafri MS High-accuracy prediction of colorectal cancer chemotherapy efficacy using machine learning applied to gene expression data. Front. Physiol. 2024, 14, 1272206. [PubMed: 38304289]

26. Gharaibeh R Z; Fodor A A; Gibas C J Background correction using dinucleotide affinities improves the performance of GCRMA. BMC Bioinform. 2008, 9, 452.

27. Irizarry R A; Bolstad B M; Collin F; Cope L M; Hobbs B; Speed TP Summaries of Affymetrix GeneChip probe level data. Nucleic Acids Res. 2003, 31, e15. [PubMed: 12582260]

28. Irizarry R A; Warren D; Spencer F; Kim I F; Biswal S; Frank B C; Gabrielson E; Garcia J G; Geoghegan J; Germino G; et al. Multiple-laboratory comparison of microarray platforms. Nat. Methods 2005, 2, 345-350. [PubMed: 15846361]

29. Tilford C A; Siemers NO Gene set enrichment analysis. Methods Mol. Biol. 2009, 563, 99-121.

### [PubMed: 19597782]

30. Kauffmann A; Gentleman R; Huber W arrayQualityMetrics—A bioconductor package for quality assessment of microarray data. Bioinformatics 2009, 25, 415-416. [PubMed: 19106121]

31. Kauffmann A; Huber W Microarray data quality control improves the detection of differentially expressed genes. Genomics 2010, 95, 138-142. [PubMed: 20079422]

32. Tweedie S; Braschi B; Gray K; Jones T E; Seal R L; Yates B; Bruford E A Genenames. org:

The HGNC and VGNC resources in 2021. Nucleic Acids Res. 2021, 49, D939-D946. [PubMed: 33152070]

33. Braschi B; Seal R L; Tweedie S; Jones T E; Bruford E A The risks of using unapproved gene symbols. Am. J. Hum. Genet. 2021, 108, 1813-1816. [PubMed: 34626580]

34. Carlson M R; Pagès H; Arora S; Obenchain V; Morgan M Genomic Annotation Resources in

R/Bioconductor. Methods Mol. Biol. 2016, 1418, 67-90. [PubMed: 27008010]

35. Cheadle C; Vawter M P; Freed W J; Becker K G Analysis of microarray data using Z score transformation. J. Mol. Diagn. 2003, 5, 73-81. [PubMed: 12707371]

36. Yasrebi H Comparative study of joint analysis of microarray gene expression data in survival prediction and risk assessment of breast cancer patients. Brief. Bioinform. 2016, 17, 771-785.

### [PubMed: 26504096]

37. Leek J T; Johnson W E; Parker HS; Jaffe A E; Storey J D The sva package for removing batch effects and other unwanted variation in high-throughput experiments. Bioinformatics 2012, 28, 882-883. [PubMed: 22257669]

38. Jung Y; Hu J A K-fold Averaging Cross-validation Procedure. J. Nonparametr. Stat. 2015, 27, 167-179. [PubMed: 27630515]

39. Kairalla J A; Coffey C S; Muller KE GLUMIP 2.0: SAS/IML Software for Planning Internal Pilots.

J. Stat. Softw. 2008, 28, 1-32. [PubMed: 27774042]

40. Ritchie M E; Phipson B; Wu D; Hu Y; Law C W; Shi W; Smyth G K limma powers differential expression analyses for RNA-sequencing and microarray studies. Nucleic Acids Res. 2015, 43, e47. [PubMed: 25605792]

41. Friedman J; Hastie T; Tibshirani R Regularization paths for generalized linear models via coordinate descent. J. Stat. Softw. 2010, 33, 1. [PubMed: 20808728]

42. Ghosh Roy G; Geard N; Verspoor K; He S PoLoBag: Polynomial Lasso Bagging for signed gene regulatory network inference from expression data. Bioinformatics 2021, 36, 5187-5193. [PubMed: 32697830]

43. Hua J LAK: Lasso and K-Means Based Single-Cell RNA-Seq Data Clustering Analysis. IEEE Access 2020, 8, 129679-129688.

44. Diaz-Uriarte R GeneSrF and varSelRF: A web-based tool and R package for gene selection and classification using random forest. BMC Bioinform. 2007, 8, 328.

45. Rigatti S J Random Forest. J. Insur. Med. 2017, 47, 31-39. [PubMed: 28836909]

46. Meyer D Support Vector Machines The Interface to libsvm in package e1071. R. News 2001, 1, 23-26.

47. He Z; Liu Z; Gong L Biomarker identification and pathway analysis of rheumatoid arthritis based on metabolomics in combination with ingenuity pathway analysis. Proteomics 2021, 21, 2100037.

48. Ferriss J S; Kim Y; Duska L; Birrer M; Levine D A; Moskaluk C; Theodorescu D; Lee J K Multi-Gene Expression Predictors of Single Drug Responses to Adjuvant Chemotherapy in Ovarian Carcinoma: Predicting Platinum Resistance. PLOS ONE 2012, 7, e30550. [PubMed: 22348014]

49. Ortiz M; Wabel E; Mitchell K; Horibata S Mechanisms of chemotherapy resistance in ovarian cancer. Cancer Drug Resist. 2022, 5, 304-316. [PubMed: 35800369]

50. Zhou J; Kang Y; Chen L; Wang H; Liu J; Zeng S; Yu L The Drug-Resistance Mechanisms of Five Platinum-Based Antitumor Agents. Front. Pharmacol. 2020, 11, 343 .[PubMed: 32265714]

51. Mondal P; Meeran S M Emerging role of non-coding RNAs in resistance to platinum-based anti-cancer agents in lung cancer. Front. Pharmacol. 2023, 14, 1105484. [PubMed: 36778005]

52. Basu A; Krishnamurthy S Cellular Responses to Cisplatin-Induced DNA Damage. J. Nucleic Acids 2010, 2010, 201367. [PubMed: 20811617]

53. Sazonova E V; Kopeina G S; Imyanitov E N; Zhivotovsky B Platinum drugs and taxanes: Can we overcome resistance? Cell Death Discov. 2021, 7, 155. [PubMed: 34226520]

54. Cummings M; Freer C; Orsi N M Targeting the tumour microenvironment in platinum-resistant ovarian cancer. Semin. Cancer Biol. 2021, 77, 3-28. [PubMed: 33607246]

55. Londero A P; Orsaria M; Tell G; Marzinotto S; Capodicasa V; Poletto M; Vascotto C; Sacco C; Mariuzzi L Expression and Prognostic Significance of APE1/Ref-1 and NPM1 Proteins in High-Grade Ovarian Serous Cancer. Am. J. Clin. Pathol. 2014, 141, 404-414. [PubMed: 24515769]

56. Wike C L; Graves H K; Hawkins R; Gibson M D; Ferdinand M B; Zhang T; Chen Z; Hudson

D F; Ottesen J J; Poirier M G; et al. Aurora-A mediated histone H3 phosphorylation of threonine 118 controls condensin I and cohesin occupancy in mitosis. Elife 2016, 5, e11402. [PubMed: 26878753]

57. Coughlan A Y; Testa G Exploiting epigenetic dependencies in ovarian cancer therapy. Int. J. Cancer 2021, 149, 1732-1743. [PubMed: 34213777]

58. Yang C; Zhang J; Ma Y; Wu C; Cui W; Wang L Histone methyltransferase and drug resistance in cancers. J. Exp. Clin. Cancer Res. 2020, 39, 173. [PubMed: 32859239]

59. Wang S; Yin C; Zhang Y; Zhang L; Tao L; Liang W; Pang L; Fu R; Ding Y; Li F; et al. Overexpression of ICAM-1 Predicts Poor Survival in High-Grade Serous Ovarian Carcinoma: A Study Based on TCGA and GEO Databases and Tissue Microarray. Biomed. Res. Int. 2019, 2019, 2867372. [PubMed: 31312656]

60. Zhan SJ; Liu B; Linghu H Identifying genes as potential prognostic indicators in patients with serous ovarian cancer resistant to carboplatin using integrated bioinformatics analysis. Oncol. Rep. 2018, 39, 2653-2663. [PubMed: 29693178]

61. Katsha A; Belkhiri A; Goff L; El-Rifai W Aurora kinase A in gastrointestinal cancers: Time to target. Mol. Cancer 2015, 14, 106. [PubMed: 25987188]

62. Du R; Huang C; Liu K; Li X; Dong Z Targeting AURKA in Cancer: Molecular mechanisms and opportunities for Cancer therapy. Mol. Cancer 2021, 20, 15. [PubMed: 33451333]

63. Buckanovich RJ; Sasaroli D; O'Brien-Jenkins A; Botbyl J; Hammond R; Katsaros D; Sandaltzopoulos R; Liotta L A; Gimotty P A; Coukos G Tumor vascular proteins as biomarkers in ovarian cancer. J. Clin. Oncol. 2007, 25, 852-861. [PubMed: 17327606]

64. Peng S.-q.; Zhu X.-r.; Zhao M.-z.; Zhang Y.-f.; Wang A.-r.; Chen M.-b.; Ye Z.-y. Identification of matrix-remodeling associated 5 as a possible molecular oncotarget of pancreatic cancer. Cell Death Dis. 2023, 14, 157. [PubMed: 36828810]

65. Minafra L; Bravatà V; Forte G I; Cammarata F P; Gilardi M C; Messa C Gene expression profiling of epithelial-mesenchymal transition in primary breast cancer cell culture. Anticancer Res. 2014, 34, 2173-2183. [PubMed: 24778019]

66. Yin L; Wang Y Long non-coding RNA NEAT1 facilitates the growth, migration, and invasion of ovarian cancer cells via the let-7 g/MEST/ATGL axis. Cancer Cell Int. 2021, 21, 437. [PubMed: 34416900]

67. Yong W; Yu D; Jun Z; Yachen D; Weiwei W; Midie X; Xingzhu J; Xiaohua W Long noncoding RNA NEAT1, regulated by LIN28B, promotes cell proliferation and migration through sponging miR-506 in high-grade serous ovarian cancer. Cell Death Dis. 2018, 9, 861. [PubMed: 30154460]

68. Chen Z J; Zhang Z; Xie B B; Zhang H Y Clinical significance of up-regulated IncRNA NEAT1 in prognosis of ovarian cancer. Eur. Rev. Med. Pharmacol. Sci. 2016, 20, 3373-3377. [PubMed: 27608895]

69. Knutsen E; Harris A L; Perander M Expression and functions of long non-coding RNA NEAT1 and isoforms in breast cancer. Br. J. Cancer 2022, 126, 551-561. [PubMed: 34671127]

70. Wang X; Shi J; Huang M; Chen J; Dan J; Tang Y; Guo Z; He X; Zhao Q TUBB2B facilitates progression of hepatocellular carcinoma by regulating cholesterol metabolism through targeting HNF4A/CYP27A1. Cell Death Dis. 2023, 14, 179. [PubMed: 36872411]

71. Kwon H; Oh S; Jin X; An Y J; Park S Cancer metabolomics in basic science perspective. Arch. Pharmacal Res. 2015, 38, 372-380.

72. Shin S-J; Chung H; Kim JY; Kim H; Cho C-H; Ha E Abstract 5748: Downregulation of glycine decarboxylase renders ovarian cancer cells less proliferative and more chemoresistant. Cancer Res. 2018, 78, 5748.

73. Fang Z; Xu S; Xie Y; Yan W Identification of a prognostic gene signature of colon cancer using integrated bioinformatics analysis. World J. Surg. Oncol. 2021, 19, 13. [PubMed: 33441161]

74. Li J; Fan H; Zhou X; Xiang Y; Liu Y Prognostic Significance and Gene Co-Expression Network of PLAU and PLAUR in Gliomas. Front. Oncol. 2022, 11, 602321. [PubMed: 35087738]

75. Zhai Y; Lu Q; Lou T; Cao G; Wang S; Zhang Z MUC16 affects the biological functions of ovarian cancer cells and induces an antitumor immune response by activating dendritic cells. Ann. Transl. Med. 2020, 8, 1494. [PubMed: 33313239]

76. Felder M; Kapur A; Gonzalez-Bosquet J; Horibata S; Heintz J; Albrecht R; Fass L; Kaur J; Hu K; Shojaei H; et al. MUC16 (CA125): Tumor biomarker to cancer therapy, a work in progress. Mol. Cancer 2014, 13, 129. [PubMed: 24886523]

77. Lakshmanan I; Ponnusamy M P; Das S; Chakraborty S; Haridas D; Mukhopadhyay P; Lele

S M; Batra S K MUC16 induced rapid G2/M transition via interactions with JAK2 for increased proliferation and anti-apoptosis in breast cancer cells. Oncogene 2012, 31, 805-817. [PubMed: 21785467]

78. Abedini M R; Wang P-W; Huang Y-F; Cao M; Chou C-Y; Shieh D-B; Tsang B K Cell fate regulation by gelsolin in human gynecologic cancers. Proc. Natl. Acad. Sci. USA 2014, 111, 14442-14447. [PubMed: 25246592]

79. Arentz G; Mittal P; Klingler-Hoffmann M; Condina M R; Ricciardelli C; Lokman N A; Kaur G; Oehler M K; Hoffmann P Label-Free Quantification Mass Spectrometry Identifies Protein Markers of Chemotherapy Response in High-Grade Serous Ovarian Cancer. Cancers 2023, 15, 2172.

### [PubMed: 37046833]

80. Kim S I; Hwangbo S; Dan K; Kim H S; Chung H H; Kim J-W; Park N H; Song Y-S; Han D; Lee

M Proteomic Discovery of Plasma Protein Biomarkers and Development of Models Predicting Prognosis of High-Grade Serous Ovarian Carcinoma. Mol. Cell. Proteom. 2023, 22, 100502.

81. Onuma T; Asare-Werehene M; Yoshida Y; Tsang BK Exosomal Plasma Gelsolin Is an Immunosuppressive Mediator in the Ovarian Tumor Microenvironment and a Determinant of Chemoresistance. Cells 2022, 11, 3305. [PubMed: 36291171]

82. Zhang W; Wang Y Activation of RIPK2-mediated NODI signaling promotes proliferation and invasion of ovarian cancer cells via NF-κB pathway. Histochem. Cell Biol. 2022, 157, 173-182. [PubMed: 34825931]

83. Velloso F J; Campos A R; Sogayar M C; Correa R G Proteome profiling of triple negative breast cancer cells overexpressing NOD1 and NOD2 receptors unveils molecular signatures of malignant cell proliferation. BMC Genom. 2019, 20, 152.

84. Abedini M R; Muller E J; Bergeron R; Gray D A; Tsang B K Akt promotes chemoresistance in human ovarian cancer cells by modulating cisplatin-induced, p53-dependent ubiquitination of FLICE-like inhibitory protein. Oncogene 2010, 29, 11-25. [PubMed: 19802016]

85. Abedini M R; Muller E J; Brun J; Bergeron R; Gray D A; Tsang BK Cisplatin Induces p53-Dependent FLICE-Like Inhibitory Protein Ubiquitination in Ovarian Cancer Cells. Cancer Res. 2008, 68, 4511-4517. [PubMed: 18559494]

86. Phippen N T; Bateman N W; Wang G; Hamilton C A; Maxwell G L; Darcy K M; Conrads T P Abstract 4632: Poor survival associated with NUAK1 overexpression in serous ovarian cancer may be explained by chemotherapy resistance. Cancer Res. 2015, 75, 4632.

87. Hou X; Liu J E; Liu W; Liu C Y; Liu Z Y; Sun Z Y A new role of NUAK1: Directly phosphorylating p53 and regulating cell proliferation. Oncogene 2011, 30, 2933-2942. [PubMed: 21317932]

88. Oh C-K; Park J J; Ha M; Heo H J; Kang J; Kwon E J; Kang J W; Kim Y; Kang J M; Yoon S Z; et al. LRRC17 Is Linked to Prognosis of Ovarian Cancer Through a p53-dependent Anti-apoptotic Function. Anticancer Res. 2020, 40, 5601-5609. [PubMed: 32988884]

89. Kidokoro T; Tanikawa C; Furukawa Y; Katagiri T; Nakamura Y; Matsuda K CDC20, a potential cancer therapeutic target, is negatively regulated by p53. Oncogene 2008, 27, 1562-1571.

### [PubMed: 17873905]

90. Xi X; Cao T; Qian Y; Wang H; Ju S; Chen Y; Chen T; Yang J; Liang B; Hou S CDC20 is a novel biomarker for improved clinical predictions in epithelial ovarian cancer. Am. J. Cancer Res. 2022, 12, 3303-3317. [PubMed: 35968331]

91. Liu C; Barger C J; Karpf AR FOXM1: A Multifunctional Oncoprotein and Emerging Therapeutic Target in Ovarian Cancer. Cancers 2021, 13, 3065. [PubMed: 34205406]

92. Guo X; Song C; Fang L; Li M; Yue L; Sun Q FLRT2 functions as Tumor Suppressor gene inactivated by promoter methylation in Colorectal Cancer. J. Cancer 2020, 11, 7329-7338. [PubMed: 33193897]

93. Vivier E; Nunès JA; Vély F Natural killer cell signaling pathways. Science 2004, 306, 1517-1519. [PubMed: 15567854]

94. Gonzalez V D; Huang Y-W; Delgado-Gonzalez A; Chen S-Y; Donoso K; Sachs K; Gentles A J; Allard G M; Kolahi K S; Howitt B E; et al. High-grade serous ovarian tumor cel modulate NK cell function to create an immune-tolerant microenvironment. Cell Rep. 2021, 36, 109632. [PubMed: 34469729]

95. Parihar R; Dierksheide J; Hu Y; Carson W E IL-12 enhances the natural killer cell cytokine response to Ab-coated tumor cells. J. Clin. Investig. 2002, 110, 983-992. [PubMed: 12370276]

96. Rao Z; Ding Y Ubiquitin pathway and ovarian cancer. Curr. Oncol. 2012, 19, 324-328. [PubMed: 23300358]

97. Sojka DR; Abramowicz A; Adamiec-Organiściok M; Karnas E; Mielańczyk Ł; Kania D; Blamek S; Telka E; Scieglinska D Heat shock protein A2 is a novel extracellular vesicle-associated protein. Sci. Rep. 2023, 13, 4734. [PubMed: 36959387]

98. Hoter A; Naim HY Heat Shock Proteins and Ovarian Cancer: Important Roles and Therapeutic Opportunities. Cancers 2019, 11, 1389. [PubMed: 31540420]

99. Wang Y; Liu Y; Liu H; Zhang Q; Song H; Tang J; Fu J; Wang X FcGBP was upregulated by

HPV infection and correlated to longer survival time of HNSCC patients. Oncotarget 2017, 8, 86503-86514. [PubMed: 29156811]

100. Koizume S; Miyagi Y Potential Coagulation Factor-Driven Pro-Inflammatory Responses in Ovarian Cancer Tissues Associated with Insufficient O2 and Plasma Supply. Int. J. Mol. Sci. 2017, 18, 809. [PubMed: 28417928]

101. Koizume S; Miyagi Y Tissue Factor-Factor VII Complex as a Key Regulator of Ovarian Cancer Phenotypes. Biomark. Cancer 2015, 7, BIC-S29318.

102. Miyake R; Yamada Y; Yamanaka S; Kawaguchi R; Ootake N; Myoba S; Kobayashi H Tissue factor pathway inhibitor 2 as a serum marker for diagnosing sasymptomatic venous thromboembolism in patients with epithelial ovarian cancer and positive D-dimer results. Mol. Clin. Oncol. 2022, 16, 46. [PubMed: 35003744]

103. Wang X; Wang E; Kavanagh JJ; Freedman RS Ovarian cancer, the coagulation pathway, and inflammation. J. Transl. Med. 2005, 3, 25. [PubMed: 15969748]

104. Judson P L; Watson J M; Gehrig P A; Fowler W C Jr.; Haskill J S Cisplatin Inhibits Paclitaxel-induced Apoptosis in Cisplatin-resistant Ovarian Cancer Cell Lines: Possible Explanation for Failure of Combination Therapy1. Cancer Res. 1999, 59, 2425-2432. [PubMed: 10344753]

105. Choi H S; Kim Y-K; Hwang K-G; Yun P-Y Increased FOXM1 Expression by Cisplatin Inhibits Paclitaxel-Related Apoptosis in Cisplatin-Resistant Human Oral Squamous Cell Carcinoma (OSCC) Cell Lines. Int. J. Mol. Sci. 2020, 21, 8897. [PubMed: 33255409]

106. Fonti V; Belitser E Feature selection using lasso. VU Amst. Res. Pap. Bus. Anal. 2017, 30, 1-25.

107. Speiser J L; Miller M E; Tooze J; Ip E A comparison of random forest variable selection methods for classification prediction modeling. Expert Syst. Appl. 2019, 134, 93-101. [PubMed: 32968335]

What is claimed:

1. A method for predicting the response of a serous ovarian cancer (SOC) patient to platinum-paclitaxel chemotherapy said method comprising the following steps:
(i) determining in a sample isolated from said patient the expression levels of one or more biomarkers, or their expression product, wherein the biomarker is the transcript, or protein product, of one or more genes selected from the group consisting of: ICAM1, TUBB2A, GLDC, PLAU, AURKA, NEAT1, MXRA5, GSN, and MUC16; and
(ii) comparing the expression levels of said one or more genes obtained in step (i) with established expression levels for responders and/or non-responders wherein similarities in the established expression levels for either responders or non-responders are indicative of whether the patient will respond to platinum-paclitaxel chemotherapy.

2. The method of claim 1, wherein the expression level of the one or more biomarker transcripts is determined through the use of a polymerase chain reaction.

3. The method of claim 1, wherein the expression level of the one or more biomarker transcripts is determined through the use of a probe that is complementary to and binds to the biomarker transcript.

4. The method of claim 1, wherein the expression level of the one or more biomarker protein products is determined through the use of an immunoassay.

5. The method of claim 1, wherein the expression level of the one or more biomarker transcript, or protein product, is determined through the use of a microarray.

6. The method of claim 1, wherein comparing the expression levels of said one or more genes obtained in step (i) comprises:
computing a prediction score based on the respective expression levels of said one or more genes, the prediction score being a weighted sum of the respective expression levels.

7. The method of claim 1, wherein comparing the expression levels of said one or more genes obtained in one of (i) comprises:
processing the respective expression levels of said one or more genes by a trained machine learning model to provide an output indicative of responsiveness or non-responsiveness to platinum-paclitaxel chemotherapy treatment.

8. A method for predicting the response of an ovarian cancer patient (SOC) patient to platinum-only chemotherapy said method consisting of:
(i) determining in a sample isolated from said patient the expression levels of one or more biomarkers, or their expression product, in a patient sample, wherein the biomarker is the transcript, or protein product, of one or more genes selected from the group consisting of: FCGBP, TFPI, NUAK1, LRRC17, FLRT2, IL12A, HSPA2, CDC20, FOXM1, and MAP4K2; and
(ii) comparing the expression levels of said one or more genes obtained in step (i) with established expression levels for responders and/or non-responders wherein similarities in the established expression levels for either responders or non-responders are indicative of whether the patient will respond to platinum-only chemotherapy treatment.

9. The method of claim 8, wherein the expression level of the one or more biomarker transcripts is determined through the use of a polymerase chain reaction.

10. The method of claim 8, wherein the expression level of the one or more biomarker transcripts is determined through the use of a probe that is complementary to and binds to the biomarker transcript.

11. The method of claim 8, wherein the expression level of the one or more biomarker protein products is determined through the use of an immunoassay.

12. The method of claim 8, wherein the expression level of the one or more biomarker transcript, or protein product, is determined through the use of a microarray.

13. The method of claim 8, wherein comparing the expression levels of said one or more genes obtained in step (i) comprises:
computing a prediction score based on the respective expression levels of said one or more genes, the prediction score being a weighted sum of the respective expression levels.

14. The method of claim 8, wherein comparing the expression levels of said one or more genes obtained in step (i) comprises:
processing the respective expression levels of said one or more genes by a trained machine learning model to provide an output indicative of responsiveness or non-responsiveness to platinum-only chemotherapy.

15. A microarray for predicting the response of a serous ovarian cancer (SOC) patient to platinum-paclitaxel chemotherapy or platinum-only chemotherapy said microarray comprising one or more probes corresponding to a group of biomarkers selected from the group consisting of:
(i) ICAM1, TUBB2A, GLDC, PLAU, AURKA, NEAT1, MXRA5, GSN, and MUC16; and/or
(ii) FCGBP, TFPI, NUAK1, LRRC17, FLRT2, IL12A, HSPA2, CDC20, FOXM1, and MAP4K2.

16. The microarray of claim 15, wherein the probe is nucleic acid molecule that is complementary to and binds to a biomarker transcript.

17. The microarray of claim 15, wherein the probe is an antibody, or fragment thereof, that binds to a biomarker protein product.

18. A test kit for predicting the response of a ovarian cancer patient (SOC) patient to platinum-paclitaxel chemotherapy or platinum-only chemotherapy comprising a group of one or more probes for measuring the expression level of one or more biomarkers said biomarkers selected from the groups consisting of:
(i) ICAM1, TUBB2A, GLDC, PLAU, AURKA, NEAT1, MXRA5, GSN, and MUC16; and
(ii) FCGBP, TFPI, NUAK1, LRRC17, FLRT2, IL12A, HSPA2, CDC20, FOXM1, and MAP4K2; and
wherein, optionally, the probe is selected from one or more of the group consisting of (a) a nucleic acid molecule that is complementary to and binds to a biomarker transcript and (b) a probe is an antibody, or fragment thereof, that binds to a biomarker protein product.

19. The method of claim 1, further comprising the step of creating a report summarizing the data obtained by analysis of the expression levels of one or more biomarkers.

20. The method of claim 8, further comprises the step of creating a report summarizing the data obtained by analysis of the expression levels of one or more biomarkers.
